(19) United States
(12) Patent Application Publication
(10) Pub. No.: US 2024/0016850 A1
(43) Pub. Date: Jan. 18, 2024

(54) CELL-NANOPARTICLE DRUG DELIVERY SYSTEM AND USE OF THE SAME FOR INHIBITING GROWTH OF TUMOR CELLS AND DIAGNOSING TUMOR CELLS

(71) Applicant: National Yang Ming Chiao Tung University, Hsinchu City (TW)

(72) Inventors: Yen-Ho Lai, Hsinchu City (TW); San-Yuan Chen, Hsinchu City (TW); Woei-Cherng Shyu, Taipei City (TW); Chih-Sheng Chiang, Taichung City (TW); Hung-Wei Cheng, New Taipei City (TW)

(21) Appl. No.: 18/353,440

(22) Filed: Jul. 17, 2023

Related U.S. Application Data
(60) Provisional application No. 63/390,061, filed on Jul. 18, 2022.

(51) Int. Cl. A61K 35/28; A61K 47/69; G01N 24/08; A61K 47/02; A61K 9/00; A61P 35/00; A61K 41/00; B82Y 5/00

(52) U.S. Cl. CPC A61K 35/28; A61K 47/6939; G01N 24/08; A61K 47/02; A61K 9/0019; A61P 35/00; A61K 41/009; B82Y 5/00

(57) ABSTRACT

A cell-nanoparticle drug delivery system includes mesenchymal stem cells and gadolinium-based agent-loaded magnetic nanoparticles which are internalized into the mesenchymal stem cells. Each of the gadolinium-based agent-loaded magnetic nanoparticles includes a core that is loaded with gadolinium-based agent and that includes a fucoidan-based inner core layer with the fucoidan non-covalently bound to the gadolinium-based agent, and a shell which includes superparamagnetic iron oxide-based inner shell layer with the superparamagnetic iron oxide bound to the gadolinium-based agent through electrical attraction, and an outer shell layer made of fucoidan and polyvinyl alcohol. Methods for inhibiting the growth of tumor cells and diagnosing the tumor cells in a subject using the cell-nanoparticle drug delivery system are also provided.

### CROSS-REFERENCE TO RELATED APPLICATION

This application claims the benefit of U.S. Provisional Patent Application No. 63/390,061, filed on Jul. 18, 2022, which is incorporated by reference herein in its entirety.

### FIELD

The present disclosure relates to a cell-nanoparticle drug delivery system. The present disclosure also relates to use of the drug delivery system for inhibiting growth of tumor cells and diagnosing tumor cells.

### BACKGROUND

Glioblastoma multiforme (GBM) is the most aggressive and most common type of cancer that originates in the brain, and has very poor prognosis for survival due to inevitable recurrence in most cases after treatment. Recent studies have demonstrated that boron neutron capture therapy, in which ¹⁰B-containing agents are selectively concentrated in GBM cells and then subjecting the resultant ¹⁰B-containing agents-concentrated GBM cells to thermal neutron beam irradiation and capture, can extend the survival in patients diagnosed with GBM. However, the efficacy of boron-neutron capture therapy has been compromised by the non-specific biodistribution and rapid metabolism of the ¹⁰B-containing agents.

In recent decades, gadolinium-neutron capture therapy has been developed to address the deficiencies of boron-neutron capture therapy including the short range of high-linear energy transfer heavy particles and the lack of dose tracking. Gadolinium agent provides a neutron capture cross-section which is approximately 67-fold higher than that of ¹⁰B, and gamma rays and internal convergent electrons emitted from gadolinium-neutron capture therapy have penetration ability which is stronger than that of the alpha particles released in boron-neutron capture therapy, enabling a homogeneous energy disposition within a tumor. In addition, gadolinium agent can be used as T1-weighted magnetic resonance imaging contrast agent to locate the tumor and track the dose distribution in real time; however, gadolinium agent has a high clearance rate, i.e., half-life of approximately two hours, necessitating a long time period of infusion of up to several hours so as to achieve the required dose at the tumor.

There are reports on implementation of nanotechnology to improve pharmacokinetics of gadolinium agent and to achieve an appropriate tumor-to-blood (T/B) ratio. For example, Verry C. et al., in an article entitled “MRI-guided clinical 6-MV radiosensitization of glioma using a unique gadolinium-based nanoparticles injection” published in Nanomedicine, 2016, Vol. 11, p. 2405-2417, discloses that AGuIX, which are ultra-small gadolinium-containing nanoparticles, show strong tumor specificity, and are capable of achieving radioenhancement in treatment of GBM. Since AGuIX are rapidly metabolized, there would be limitations in applications of such nanoparticles in gadolinium-neutron capture therapy.

Targeted delivery of gadolinium agent to GBM tumor cells may be achieved using umbilical cord-derived mesenchymal stem cells (UMSCs) which serve as a cellular vehicle that is capable of penetrating the blood-brain barrier. Sonabend A. M. et al., in an article entitled “Mesenchymal stem cells (MSCs) effectively deliver an oncolytic adenovirus to intracranial glioma” published in Stem Cells, 2008, Vol. 26, p. 831-841, discloses that use of MSCs to deliver an oncolytic adenovirus to glioma cells resulted in a 46-fold increase in viral copies in the glioma cells. However, direct internalization of gadolinium agent into the UMSCs may lead to gadolinium ions (i.e., Gd³⁺) being released from the gadolinium agent before the UMSCs reached the targeted tumor, causing toxicity and impairing cellular function of the UMSCs, thereby reducing the efficiency of targeted delivery of the gadolinium agent.

Therefore, there is an urgent need to develop a new strategy which utilizes nanoparticles and mesenchymal stem cells to precisely deliver gadolinium agent to GBM cells for effective treatment.

### SUMMARY

Therefore, an object of the present disclosure is to provide a cell-nanoparticle drug delivery system and methods for inhibiting the growth of tumor cells and diagnosing the tumor cells using the drug delivery system which can alleviate at least one of the drawbacks of the prior art.

According to one aspect of the present disclosure, the cell-nanoparticle drug delivery system includes mesenchymal stem cells and gadolinium-based agent-loaded magnetic nanoparticles which are internalized into the mesenchymal stem cells. Each of the gadolinium-based agent-loaded magnetic nanoparticles includes a core and a shell. The core is loaded with gadolinium-based agent and includes a fucoidan-based inner core layer with the fucoidan non-covalently bound to the gadolinium-based agent. The shell includes a superparamagnetic iron oxide-based inner shell layer with the superparamagnetic iron oxide bound to the gadolinium-based agent through electrical attraction, and an outer shell layer made of fucoidan and polyvinyl alcohol.

According to another aspect of the present disclosure, the method for inhibiting the growth of tumor cells in a subject includes administering to the subject the aforesaid cell-nanoparticle drug delivery system by injection, navigating the cell-nanoparticle drug delivery system to the tumor cells of the subject using an external magnetic field, and subjecting the tumor cells of the subject to neutron beam irradiation, so that gamma rays and internal convergent electrons emit from gadolinium-based agent to kill the tumor cells.

According to yet another aspect of the present disclosure, the method for diagnosing tumor cells in a subject includes administering to the subject the aforesaid cell-nanoparticle drug delivery system by injection, navigating the cell-nanoparticle drug delivery system to the tumor cells of the subject using an external magnetic field, and subjecting the subject to magnetic resonance imaging analysis so as to locate the tumor cells.

### BRIEF DESCRIPTION OF THE DRAWINGS

Other features and advantages of the present disclosure will become apparent in the following detailed description of the embodiment(s) with reference to the accompanying drawings. It is noted that various features may not be drawn to scale.

FIG. 1 shows (a) to (c) scanning electron microscopy images of gadodiamide-loaded magnetic nanoparticles, i.e., Gd-FPFNP, Gd-FFNP and Gd-PPNP, (d) to (f) transmission electron microscopy images of Gd-FPFNP, Gd-FFNP and Gd-PPNP, and (g) energy-dispersive X-ray spectroscopy image of Gd-FPFNP of Example 1, infra.

FIG. 2 shows (a) distribution of particle size, (b) zeta potentials, (c) stability in phosphate-buffered saline (PBS), and (d) stability in PBS with fetal bovine serum, of Gd-FPFNP, Gd-FFNP and Gd-PPNP of Example 1, infra.

FIG. 3 shows (a) FTIR spectra of polyvinyl alcohol (PVA), fucoidan, and Gd-FPFNP, and (b) the magnetization-saturation curves for iron oxide (10) and Gd-FPFNP of Example 1, infra.

FIG. 4 is a schematic view illustrating the structure of Gd-FPFNP of Example 1, infra.

FIG. 5 shows cumulative amounts of gadodiamide released at different times for Gd-FFNP, Gd-PPNP and Gd-FPFNP of Example 1, infra.

FIG. 6 shows concentrations of gadolinium ions (Gd³⁺) released from gadodiamide and Gd-FPFNP at different times of Example 1, infra, in which the symbol “**” represents p<0.01 compared with the gadodiamide.

FIG. 7 shows concentrations of gadodiamide in Gd-FFNP-treated umbilical cord-derived mesenchymal stem cells (UMSCs), Gd-PPNP-treated UMSCs, and Gd-FPFNP-treated UMSCs at different times of Example 1, infra.

FIG. 8 shows a transmission electron microscopy image of a cell-nanoparticle drug delivery system of the present disclosure, i.e., Gd-FPFNP-treated UMSCs of Example 2, infra.

FIG. 9 is a flow cytometry diagram illustrating the efficiency of internalization of Gd-FPFNP into UMSCs by cellular uptake at different times of Example 2, infra.

FIG. 10 shows the cell viability of Gd-FPFNP-treated UMSCs and gadodiamide-treated UMSCs of Example 2, infra, in which the symbol “*” represents p<0.05 compared with the gadodiamide-treated UMSCs.

FIG. 11 shows (a) the MR relaxation rates of gadodiamide and Gd-FPFNP at different concentrations of gadodiamide, and (b) the MR relaxation rates of FPFNP and Gd-FPFNP at different concentrations of iron oxide of Example 2, infra.

FIG. 12 shows (a) the T1-weighted images (T1WI) of gadodiamide-treated UMSCs and cell-nanoparticle drug delivery system (CNDDS), and (b) the T2-weighted images (T2WI) of the gadodiamide-treated UMSCs and CNDDS of Example 2, infra.

FIG. 13 shows the relative fold of SDF-1α mRNA level in the brain of the rats in each group of Example 3, infra, in which the symbol “**” represents p<0.01 compared with the control group.

FIG. 14 shows magnetic resonance imaging (MRI) and bioluminescence (BLI) images of the brain of the orthotopic glioblastoma multiforme (GBM)-bearing rats at different times post-administration of the cell-nanoparticle drug delivery system of Example 3, infra.

FIG. 15 shows the contents of gadolinium in vital organs of the orthotopic GBM-bearing rats at different times post-administration of the cell-nanoparticle drug delivery system of Example 3, infra.

FIG. 16 shows the contents of gadolinium in vital organs of the orthotopic GBM-bearing rats in each group at the 24^{th} hour post-administration of the cell-nanoparticle drug delivery system of Example 3, infra.

FIG. 17 shows (a) the tumor-to-blood (T/B) ratio and (b) the tumor-to-normal tissue (T/N) ratio of the orthotopic GBM-bearing rats in each group of Example 3, infra, in which the symbol “**” represents p<0.01 compared with the comparative group 2.

FIG. 18 shows a confocal laser scanning microscopy image of a fusion progeny resulting from fusion between GBM8401 cell and UMSC of the cell-nanoparticle drug delivery system of Example 3, infra.

FIG. 19 shows the cell viability of GBM8401 cells after neutron beam irradiation in each group of Example 3, infra, in which the symbols “*” and “**” respectively represent p<0.05 and p<0.01 compared with the respective comparative groups.

FIG. 20 shows light microscopy images of hematoxylin and eosin (H&E) stain of the brains of the orthotopic GBM-bearing rats after neutron beam irradiation in each group of Example 3, infra.

FIG. 21 shows the tumor volumes of the orthotopic GBM-bearing rats in each group of Example 3, infra, in which the symbols “*” and “**” respectively represent p<0.05 and p<0.01 compared with the comparative group 1, and the symbols “#” and “##” respectively represent p<0.05 and p<0.01 between the respective groups.

FIG. 22 shows the percentages of survival for the orthotopic GBM-bearing rats in each group of Example 3, infra, in which the symbols “#” and “** ##” respectively represent p<0.05 and p<0.01 compared with the blank control group.

FIG. 23A shows body weight changes of healthy male C57BL/6 Narl mice in each group of Example 3, infra.

FIG. 23B shows H&E stain of vital organs of healthy male C57BL/6 Narl mice in each group of Example 3, infra.

FIG. 24 shows differences in serum levels of pro-inflammatory factors and those of anti-inflammatory cytokines in the orthotopic GBM-bearing rats of each group of Example 4, infra, in which the symbols “*” and “**” respectively represent p<0.05 and p<0.01 compared with the control group.

### DETAILED DESCRIPTION

Before the present disclosure is described in greater detail, it should be noted that if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In order to address the current limitations of gadolinium-neutron capture therapy in the inhibition of tumor, the applicants endeavored to develop improved methods and found that a cell-nanoparticle drug delivery system prepared by integrating mesenchymal stem cells and magnetic nanoparticles loaded with a gadolinium-based agent, is not only physiologically stable, but also can be magnetically navigated towards the site of the tumor so as to inhibit growth of the tumor cells without damaging the adjacent normal tissues.

As used herein, the term “a gadolinium-based agent” refers to gadolinium-containing molecular complexes which are used for enhancement of vessels in magnetic resonance angiography and/or for brain tumor enhancement.

Therefore, the present disclosure provides a cell-nanoparticle drug delivery system which includes mesenchymal stem cells and gadolinium-based agent-loaded magnetic nanoparticles which are internalized into the mesenchymal stem cells. Each of the gadolinium-based agent-loaded magnetic nanoparticles includes a core and a shell. The core is loaded with gadolinium-based agent and includes a fucoidan-based inner core layer with the fucoidan non-covalently bound to the gadolinium-based agent. The shell includes a superparamagnetic iron oxide-based inner shell layer with the superparamagnetic iron oxide bound to the gadolinium-based agent through electrical attraction, and an outer shell layer made of fucoidan and polyvinyl alcohol.

Examples of the gadolinium-based agent include, but are not limited to, gadoterate, gadobutrol, gadoteridol, gadopentetate, gadobenate, gadopentetic acid, gadoxentate, gadoversetamide, and gadodiamide. In certain embodiments, the gadolinium-based agent is gadodiamide.

According to the present disclosure, the fucoidan of the fucoidan-based inner core layer is non-covalently bound to the gadolinium-based agent through hydrophilic and hydrophobic interaction.

According to the present disclosure, the fucoidan is obtained from a brown seaweed material and has anti-inflammatory property. Examples of the brown seaweed material include, but are not limited to, Cladosiphon okamuranus, Undaria pinnatifida, Laminaria japonica, and Fucus vesiculosus. In an exemplary embodiment, the brown seaweed material is Fucus vesiculosus.

In certain embodiments, the fucoidan has an average molecular weight ranging from 1 kDa to 200 kDa.

According to the present disclosure, based on a total weight of each of the gadolinium-based agent-loaded magnetic nanoparticles, the fucoidan is present in an amount ranging from 2 wt % to 60 wt %, the superparamagnetic iron oxide is present in an amount ranging from 0.15 wt % to 20.0 wt %, and the gadolinium-based agent is present in an amount ranging from 0.5 wt % to 40.0 wt %.

According to the present disclosure, the superparamagnetic iron oxide may have a concentration ranging from 1 mg/mL to 100 mg/mL.

In certain embodiments, each of the gadolinium-based agent-loaded magnetic nanoparticles has a particle size ranging from 50 nm to 500 nm.

According to the present disclosure, the cell-nanoparticle drug delivery system of the present disclosure is prepared by treating the mesenchymal stem cells with the gadolinium-based agent-loaded magnetic nanoparticles for a predetermined time period to allow the gadolinium-based agent-loaded magnetic nanoparticles to be internalized by cellular uptake into the mesenchymal stem cells.

In certain embodiments, the gadolinium-based agent is present in an amount ranging from 0.1 pg/cell to 100 pg/cell in the mesenchymal stem cells after the mesenchymal stem cells are treated with the gadolinium-based agent-loaded magnetic nanoparticles for a time period ranging from 1 hour to 72 hours.

In certain embodiments, the mesenchymal stem cells are selected from the group consisting of umbilical cord-derived mesenchymal stem cells, adipose-derived mesenchymal stem cells, bone marrow-derived mesenchymal stem cells, and placenta-derived mesenchymal stem cells.

In an exemplary embodiment, the mesenchymal stem cells are umbilical cord-derived mesenchymal stem cells.

In certain embodiments, the gadolinium-based agent is present in an amount ranging from 0.1 pg/cell to 30 pg/cell in the umbilical cord-derived mesenchymal stem cells after the umbilical cord-derived mesenchymal stem cells are treated with the gadolinium-based agent-loaded magnetic nanoparticles for a time period ranging from 0.5 hours to 48 hours.

The present disclosure also provides a method for inhibiting the growth of tumor cells in a subject. The method includes administering to the subject the aforesaid cell nanoparticle-drug delivery system by injection, navigating the cell-nanoparticle drug delivery system to the tumor cells of the subject using an external magnetic field, and subjecting the tumor cells of the subject to neutron beam irradiation, so that gamma rays and internal convergent electrons emit from gadolinium-based agent to kill the tumor cells.

As used herein, the term “subject” refers to any animal of interest, such as humans, monkeys, cows, sheep, horses, pigs, goats, dogs, cats, mice, and rats. In certain embodiments, the subject is a rat.

As used herein, the term “administration” or “administering” means introducing, providing or delivering a pre-determined active ingredient to a subject by any suitable routes to perform its intended function.

Examples of the tumor cells include, but are not limited to, head and neck tumor cells, brain tumor cells, skin tumor cells, pancreatic tumor cells, liver tumor cells, and lung tumor cells. An example of the head and neck tumor cells are oral tumor cells.

In an exemplary embodiment, the brain tumor cells are glioblastoma multiforme cells.

According to the present disclosure, the cell-nanoparticle drug delivery system may be formulated into a dosage form suitable for parenteral administration using technology well known to those skilled in the art.

According to the present disclosure, for parenteral administration, the cell-nanoparticle drug delivery system according to the present disclosure may be formulated into an injection, e.g., a sterile aqueous solution, a dispersion or an emulsion.

The cell-nanoparticle drug delivery system according to the present disclosure may be administered via one of the following parenteral routes: intraperitoneal injection, intrapleural injection, intramuscular injection, intravenous injection, intracarotid injection intraarterial injection, intraarticular injection, intrasynovial injection, intrathecal injection, intracranial injection, intraepidermal injection, subcutaneous injection, intradermal injection, and intralesional injection.

In certain embodiments, the cell-nanoparticle drug delivery system is administered by one of intracarotid injection and intravenous injection. In an exemplary embodiment, the cell-nanoparticle drug delivery system is administered by intracarotid injection.

Since gadolinium-based agent is a well-known magnetic resonance imaging (MRI) contrast agent, the cell-nanoparticle drug delivery system including the gadolinium-based agent-loaded magnetic nanoparticles is expected to be useful for diagnosing tumor cells.

Therefore, the present disclosure also provides a method for diagnosing tumor cells in a subject. The method includes administering to the subject the aforesaid cell-nanoparticle drug delivery system by injection, navigating the cell-nanoparticle drug delivery system to the tumor cells of the subject using an external magnetic field, and subjecting the subject to magnetic resonance imaging analysis so as to locate the tumor cells.

The dose and frequency of administration of the cell-nanoparticle drug delivery system may vary depending on the following factors: the severity of the illness or disorder to be treated, routes of administration, and age, physical condition and response of the subject to be treated. In general, the drug delivery system may be administered in a single dose or in several doses.

The present disclosure will be described by way of the following examples. However, it should be understood that the following examples are intended solely for the purpose of illustration and should not be construed as limiting the present disclosure in practice.

### EXAMPLES

### General Experimental Materials:

### 1. Chemicals

Gadodiamide (Omniscan™), which is a gadolinium (III) (Gd³⁺)-based magnetic resonance imaging (MRI) contrast agent used clinically, was obtained from China Medical University Hospital, Taichung, Taiwan. Fucoidan (extracted from Fucus vesiculosus), sodium meta-periodate, chloroform, 1,2-hexadecanediol (97%), Fe(acac)₃, oleic acid (90%), olecylamine (>70%), sodium azide, and benzyl ether (99%) were purchased from Sigma Aldrich. Polyvinyl alcohol (PVA) (mw of 25 K) was purchased from Fluka Chemical Co. Quantum dots (CdSe/ZnS with excitation/emission=612/620 nm) were purchased from Ocean NanoTech. Cy5.5 dye was purchased from Life Science Technology.

### 2. Source and Cultivation of Glioblastoma Multiforme (GBM) Cells

Human brain malignant glioma cell line GBM8401 and rat glioma cell line F98 used for establishing orthotopic GBM-bearing rats were purchased from the American Type Culture Collection (ATCC® CRL-2397™) and the Bioresource Collection and Research Centre, Taiwan (BCRC No.: 60613), respectively. These cell lines were authenticated by the respective supplier using morphology, karyotyping, or polymerase chain reaction analysis.

Luciferase-expressing F98 (F98-Luc) cells were obtained by transforming F98 cells with the luciferase gene using technology well known to those skilled in the art. In brief, luciferase cDNA from luciferase-pcDNA3 (Plasmid #18964 available from Addgene) were transferred into pIRES expression vector by specific restriction enzyme linker (EcoR1 and Nhe1) so as to obtain pSF-luciferase construct. The pPB-CMV-MCS-EF1α-Puro PiggBac vector (System Bioscience) which contains multiple cloning sites (MCS), PiggBac terminal repeats (PB-TRs), core insulators (CIs) and puromycin selection marker driven by human elongation factor (EF) 1α, was used as the base vector. DNA fragments of luciferase gene from the pSF-luciferase construct was amplified by polymerase chain reaction and sub-cloned into the pPB-CMV-MCS-EF1α-Puro PiggyBac vector at a site upstream of the coding region of EF1α, so as to obtain pPB-luciferase construct. In order to generate F98-Luc stable cells, the pPB-luciferase construct was co-transfected with a PiggyBac transposase (System Biosciences) into F98 cells using Amaxa Nucleofactor™ II/2b transfection device (Lonza), followed by selection by puromycin.

The F98 cells, F98-Luc cells and GBM8401 cells were cultivated in Dulbecco's modified Eagle's medium (DMEM purchased from Gibco) supplemented with 10% (v/v) fetal bovine serum (Gibco) and 0.5% (w/v) penicillin-streptomycin (Gibco) at 37° C. under 5% (v/v) CO₂ and 95% (v/v) air.

### 3. Source and Cultivation of Umbilical Cord Mesenchymal Stem Cells

Umbilical cord-derived mesenchymal stem cells (UMSCs) were collected from human umbilical cord tissues using protocols approved by the Institutional Review Board (IRB) of the China Medical University Hospital, Taichung, Taiwan (IRB number: CMUH-110-REC-1-068).

First, the human umbilical cord tissues were washed three times with Ca²⁺-free and Mg²⁺-free phosphate-buffered saline (DPSB purchased from Life Technology), and then was excised using scissors in a midline direction. Next, the vessels of the umbilical artery, vein and outlining membrane were separated from the Wharton's jelly of the human umbilical cord tissues, were cut into pieces each having a size smaller than 0.5 cm³, were subjected to treatment with collagenase type I (Sigma Aldrich), followed by incubation at 37° C. under humidified atmosphere containing 95% air and 5% CO₂ for 3 hours. The resultant explants were cultivated in DMEM supplemented with 10% fetal calf serum (Gibco) and 0.5% penicillin-streptomycin (Gibco) at 37° C. under humidified atmosphere containing 95% air and 5% CO₂ for 5 to 7 days to allow migration of mesenchymal stem cells from the explants. The thus obtained UMSCs used in the following experiments were verified to be free from mycoplasma contamination.

### 4. Experimental Animals

The experimental animals, i.e., female F344/NNral rats (RMRC21002) and male C57BL/6 JNarl mice (RMRC11005) used in the following experiments were purchased from the National Laboratory Animal Center, Taiwan. All the experimental animals were housed in an animal room with an independent air conditioning system under the following laboratory conditions: an alternating 12-hour light and 12-hour dark cycle, a temperature maintained at 23° C.±2° C., and a relative humidity maintained at 50%±10%. The experimental animals were provided with water and fed ad libitum. All experimental procedures involving the experimental animals were approved by the Institutional Animal Care and Use Committee (IACUC) of the China Medical University (IACUC No.: CMU 2020-014), and were in compliance with the legal provision of the Animal Protection Act of Taiwan, and were carried out according to the guidelines of the Animal Care Committee of the Council of Agriculture, Taiwan.

### General Experimental Procedures:

### 1. Statistical Analysis

All the experiments described below were performed at least 3 times, unless otherwise noted. Statistical analysis was conducted using GraphPad Prism 9 software (Developer: GraphPad Software, Inc., San Diego, CA). The experimental data of all the test groups are expressed as mean±standard deviation (SD), and were analyzed using two-tailed Student's t-test or one-way ANOVA with Tukey's multiple comparison test unless otherwise noted, so as to assess the differences between the groups. Statistical significance is indicated by p<0.05.

### Example 1. Preparation and Evaluation of gadodiamide-loaded Magnetic Nanoparticles

In this example, gadodiamide-loaded magnetic nanoparticles with different configurations were prepared and then subjected to evaluations.

A. Preparation of gadodiamide-loaded Magnetic Nanoparticles

Superparamagnetic iron oxide (SPIO) nanoparticles for preparing the gadodiamide-loaded magnetic nanoparticles were synthesized using procedures described by Sun S. et al. in an article entitled “Monodisperse MFe₂O4 (M=Fe, Co, Mn) Nanoparticles” published in J. Am. Chem. Soc., 2004, Vol. 126, p. 273-279. In brief, Fe(acac)₃ (2 mmol), 1,2-hexadecanediol (10 mmol), oleic acid (6 mmol), and olecylamine (6 mmol) were mixed with benzyl ether (20 mL) in a three-necked bottle, and then subjected to a reflux reaction conducted at 100° C. for 30 minutes under a nitrogen atmosphere, followed by heating to 200° C. for 1 hour and then to 285° C. for 30 minutes, so as to complete the nucleation and growth of SPIO nanoparticles. After cooling to room temperature, the SPIO nanoparticles were collected by centrifugation at 6200×g for 10 minutes and then purified three times using ethanol.

Synthesis of gadolinium-loaded magnetic nanoparticles were conducted using an organic phase solution containing the SPIO nanoparticles, and two hydrophilic phase solutions. First, 0.2 mL of a first hydrophilic phase solution containing 2 mg of fucoidan (1 wt %) and 30 mg of gadodiamide was added to the organic phase solution containing 4 mg of SPIO nanoparticles in 0.4 mL of chloroform to form a mixture. Afterwards, the mixture was subjected to a first emulsification reaction by pulsed ultrasound sonification conducted at 120 W for 60 seconds using a homogenizer (Manufacturer: Double Eagle Enterprise Co., Ltd., Taiwan), so as to form a water-in-oil emulsion. Next, the water-in-oil emulsion and 3.0 mL of a second hydrophilic phase solution containing 15 mg of fucoidan (1 wt %) and 15 mg of PVA (1 wt %) were subjected to a second emulsification reaction by pulsed ultrasound sonification conducted at 120 W for 60 seconds using the homogenizer, so as to form a water-in-oil-in-water emulsion. Then, the water-in-oil-in-water emulsion was subjected to removal of residual chloroform thereform using an evaporator, followed by purification using MagniSort® cell separation kit (Manufacturer: e-Bioscience) and resuspension in distilled deionized water, thereby obtaining gadodiamide-loaded magnetic nanoparticles with configuration of Gd-Fu@IO@PVA/Fu (abbreviated as Gd-FPFNP).

For comparison, gadodiamide-loaded magnetic nanoparticles with configuration of Gd-Fu@IO@Fu (abbreviated as Gd-FFNP) and gadodiamide-loaded magnetic nanoparticles with configuration of Gd-PVA@IO@PVA (abbreviated as Gd-PPNP) were prepared using procedures similar to those of the Gd-FPFNP as described above, except that, for the Gd-FFNP, the second hydrophilic phase solution contained 30 mg of fucoidan without PVA, whereas for the Gd-PPNP, the second hydrophilic phase solution contained 30 mg of PVA without fucoidan.

B. Evaluation of gadodiamide-Loaded Magnetic Nanoparticles

A respective one of the Gd-FFNP, Gd-PPNP and Gd-FPFNP obtained in section A above were subjected to property evaluation described hereinafter.

### 1. Determination of Morphology, and Physical and Chemical Properties

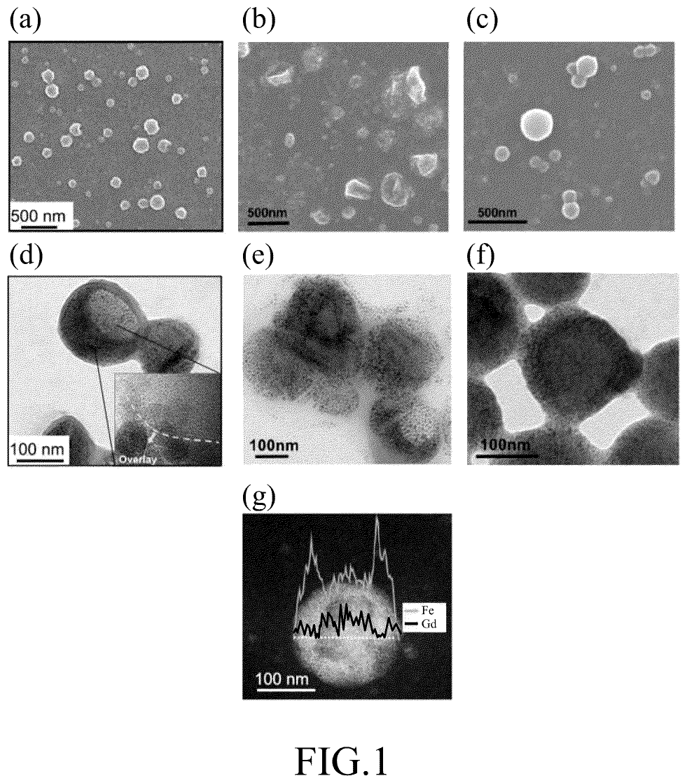

A respective one of the Gd-FPFNP, Gd-FFNP, and Gd-PPNP was subjected to observation of morphology using scanning electron microscope and transmission electron microscope (Manufacturer: Philips; Model no.: CM-120). The results are shown in FIG. 1(a) to (f). The Gd-FPFNP were further subjected to elemental mapping imaging with line scanning profile using energy-dispersive X-ray spectroscopy (EDXS). The result is shown in FIG. 1(g).

FIG. 1(a) to (c) respectively show the morphologies of Gd-FPFNP, Gd-FFNP and Gd-PPNP observed by scanning electron microscopy, while FIG. 1(d) to (f) respectively show the morphologies of Gd-FPFNP, Gd-FFNP and Gd-PPNP observed by transmission electron microscopy. As shown in FIGS. 1(b) and (e), Gd-FFNP were presented as non-spherical nanostructures and were not structurally stable because fucoidan lacks amphipathic properties to stabilize water-in-oil interface. In contrast, as shown in FIGS. 1(a), (c), (d), and (f), both Gd-FPFNP and Gd-PPNP were presented as spherical and homogenous microstructures which were attributed to the amphiphilic properties of PVA. Referring again to FIG. 1(d), the core-shell structure of Gd-FPFNP can be clearly observed by transmission electron microscopy, in which the collapsed shell was overlaid and displayed a dark contrast as shown in the inset high-resolution image.

FIG. 1(g) show the transmission electron microscopy line scan image and EDXS analysis of Gd-FPFNP. As shown in FIG. 1(g), iron (Fe) signals peaked at two ends whereas Gd signals were centralized in the core region, suggesting that in each Gd-FPFNP, the iron oxide of the SPIO nanoparticle were distributed in the shell while gadodiamide was concealed in the core.

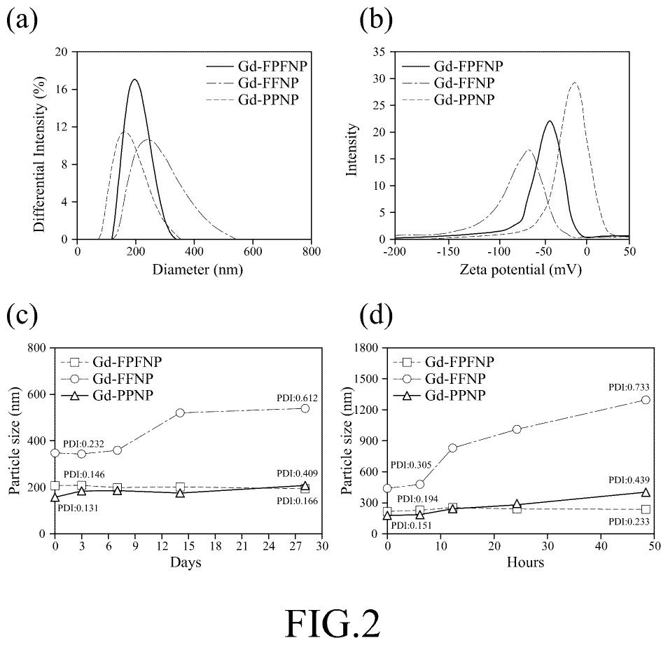

In addition, Gd-FPFNP, Gd-FFNP, and Gd-PPNP were subjected to determination of distribution of particle size, polydispersity index, zeta potential, and stability in phosphate-buffered saline (PBS) solution and in PBS solution containing 10% fetal bovine serum (FBS) (simulating blood environment) by dynamic light scattering (DLS) using Delsa C particle analyzer (Manufacturer: Beckman Coulter). The results are shown in FIG. 2(a) to (d).

FIGS. 2(a) and (b) are respectively show the distributions of particle size and zeta potentials of Gd-FPFNP, Gd-FFNP and Gd-PPNP. As shown in FIG. 2(a), Gd-FPFNP showed a wide size band, suggesting a heterogenous distribution of particle size. It should be noted that the particle size and polydispersity index of Gd-FPFNP, Gd-FFNP and Gd-PPNP were 220 nm/0.146, 385 nm/0.305, and 190 nm/0.131, respectively. The polydispersity index of Gd-FPFNP was smaller to that of Gd-FFNP, which may be attributed to the formation of hydrogen bonds between PVA and fucoidan. As shown in FIG. 2(b), in comparison to the Gd-PPNP which had neutral zeta potential, both Gd-FPFNP and Gd-FFNP exhibited a strong negative-charged surface due to the presence of sulfate groups in fucoidan. FIGS. 2(c) and (d) are graphs respectively showing changes in particle size over time in PBS solution and in PBS solution containing 10% FBS of Gd-FPFNP, Gd-FFNP and Gd-PPNP. As shown in FIGS. 2(c) and (d), Gd-FFNP had particle size which increased substantially over time and became polydisperse at day 28 in both the PBS solution and PBS solution containing 10% FBS, whereas although Gd-FPFNP and Gd-PPNP showed sustained colloidal stability in both solutions with minimal changes in particle size in the first 14 days, only Gd-FPFNP maintained a monodispersed condition at day 28 in both solutions, suggesting that Gd-FPFNP has a structure that provides steric hindrance to avoid protein opsonization and allows Gd-FPFNP to remain intact in physiological conditions.

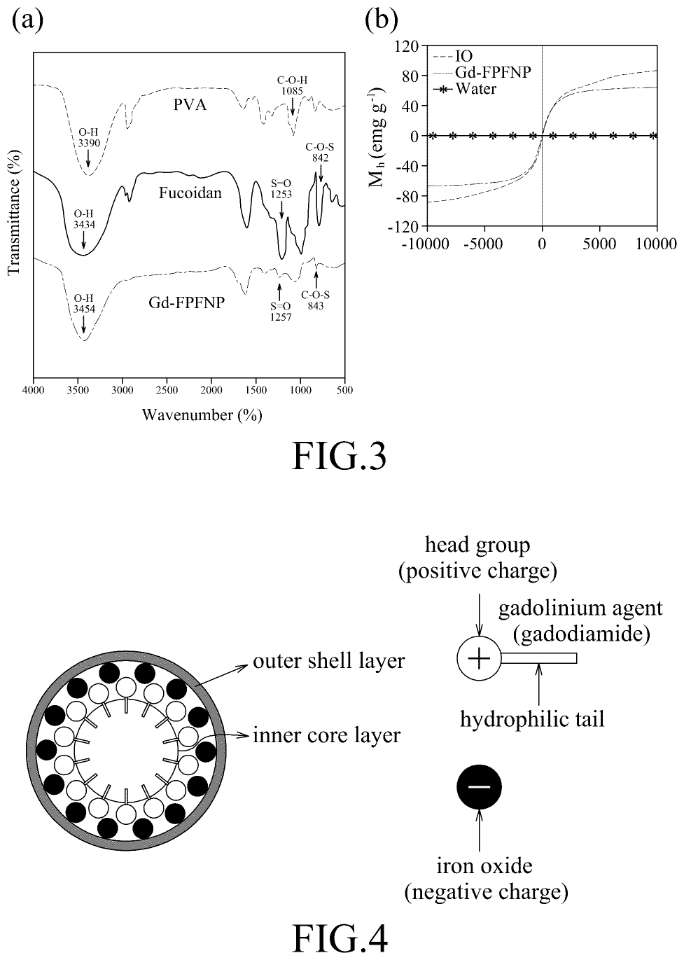

In order to confirm the presence of fucoidan, Gd-FPFNP were further subjected to Fourier-transform infrared (FTIR) spectroscopy. In addition, Gd-FPFNP were subjected to determination of magnetic properties. The results are shown in FIGS. 3(a) and (b).

FIG. 3(a) shows FTIR spectra of PVA, fucoidan, and Gd-FPFNP. As shown in FIG. 3(a), the two characteristics peaks for fucoidan are a peak at 1253 cm⁻¹ attributed to S═O antisymmetric stretching vibration of the sulfate group; and another peak at 842 cm⁻¹ attributed to C—O—S bonding where the sulfate ester was bound to the fucose. The presence of these two characteristic peaks in the FTIR spectrum of Gd-FPFNP indicates that fucoidan was present on the surface of Gd-FPFNP. FIG. 3(b) shows the magnetization-saturation curves for iron oxide (10) and Gd-FPFNP. As shown in FIG. 3(b), in comparison to water which does not have magnetic property, saturated magnetizations of 10 and Gd-FPFNP were measured as 84.7 emu/g and 73.5 emu/g, demonstrating that Gd-FPFNP retained magnetic properties of 10 after synthesis.

FIG. 4 is a schematic view illustrating the structure of Gd-FPFNP, each including a core and a shell. As shown in FIG. 4, the core is loaded with gadodiamide and includes a fucoidan-based inner core layer that is made of fucoidan, while the shell includes a superparamagnetic iron oxide-based inner shell layer that is made of iron oxide and an outer shell layer made of fucoidan and PVA. The gadodiamide loaded and concealed in the core includes a hydrophilic tail arranged inwardly which is capable of forming non-covalent bond with fucoidan, and a positively-charged head group bound to the iron oxide with a negative charge. It should be noted that the fucoidan of the fucoidan-based inner core layer is non-covalently bound to the gadodiamide through hydrophilic and hydrophobic interaction.

### 2. Determination of Loading Capacity and Encapsulation Efficiency

A respective one of the Gd-FFNP, Gd-PPNP, Gd-FPFNP and gadodiamide were lyophilized and then subjected to determination of content of gadodiamide using Agilent 7800 inductively coupled plasma mass spectrometry (ICP-MS) system. The loading capacity percentage of a respective one of the Gd-FFNP, Gd-PPNP and Gd-FPFNP was calculated using the following formula (1):

A=(B/C)×100% (1)
wherein A=loading capacity percentage (%)
   B=mass of gadodiamide
   C=mass of a respective one of Gd-FFNP, Gd-PPNP and Gd-FPFNP

The encapsulation efficiency (in percentage) of a respective one of the Gd-FFNP, Gd-PPNP and Gd-FPFNP was calculated using the following formula (2):

D=(E/F)×100% (2)
wherein D=encapsulation percentage (%)
   E=amount of gadodiamide
   F=total amount of gadodiamide used for synthesizing a respective one of Gd-FFNP, Gd-PPNP and Gd-FPFNP

The results are shown in Table 1 below.

TABLE 1

| | Loaded Gd | Encapsulation | Loading |
|---|---|---|---|
| | content (mg) | efficiency (%) | capacity (%) |
| Gd-FFNP | 13.70 | 48.93 | 38.14 |
| Gd-PPNP | 15.47 | 55.27 | 18.78 |
| Gd-FPFNP | 16.69 | 59.64 | 28.57 |

As shown in Table 1, in comparison with Gd-FFNP and Gd-PPNP, Gd-FPFNP had the highest amount of gadodiamide loaded therein, and also had the highest encapsulation efficiency and loading capacity. In comparison with Gd-FPFNP, Gd-FFNP were structurally unstable (see FIGS. 1(b) and (e)) and had 17.9% less gadodiamide loaded therein (13.70 mg vs 16.69 mg). 3. Determination of in vitro cumulative amount of gadodiamide released

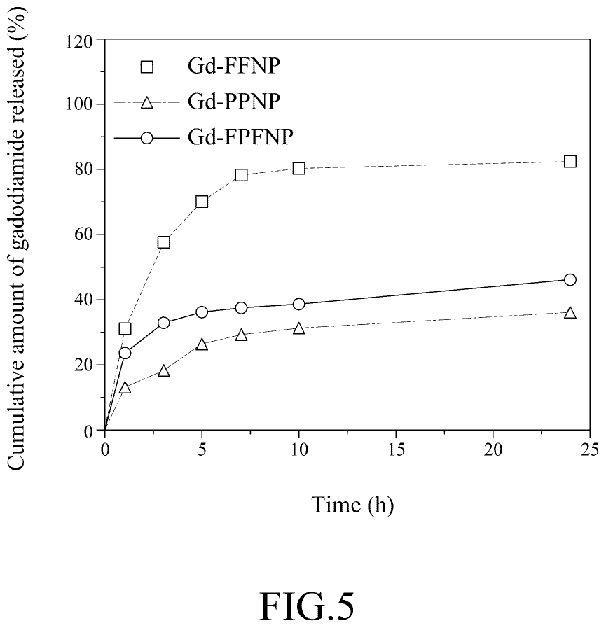

A respective one of the Gd-FFNP, Gd-PPNP and Gd-FPFNP was subjected to determination of in vitro cumulative amount of gadodiamide released at different times. In brief, the Gd-FFNP, Gd-PPNP and Gd-FPFNP were respectively added into a dialysis tube with cellulose membrane having a molecular weight cutoff of 1 kDa, followed by immersing the dialysis tube in 10 mL of phosphate-buffered saline (PBS) and stirring at 37° C. Thereafter, 0.5 mL aliquots of the PBS were taken out after 0, 5, 10, 15, 20 and 25 hours of stirring, and then subjected to high performance liquid chromatography (HPLC) using Agilent 1200 series HPLC system, so as to quantify the cumulative amounts of gadodiamide released at the predetermined time points. The HPLC was performed under the following conditions: LiChrospher® C18 column (Manufacturer: Merck) having a size of 250*4 mm, 5 μm; mobile phase aqueous solvent containing trimethylamine (15 mmol/L) and glacial acetic acid (5 mmol/L; pH 6.5 to 7.0); flow rate of 1.0 mL/minute at ambient temperature; and UV detection under a wavelength of 210 nm. The retention time of gadodiamide was 4.5 minutes. The results are shown in FIG. 5.

FIG. 5 is shows cumulative amount of gadodiamide released at different times over 24 hours for Gd-FFNP, Gd-PPNP and Gd-FPFNP. As shown in FIG. 5, Gd-FPFNP and Gd-PPNP showed slow release of gadodiamide therefrom, and the cumulative amounts of gadodiamide released for Gd-FPFNP and Gd-PPNP over 24 hours stabilized at 46% and 35%, respectively. The slow release pattern exhibited by Gd-FPFNP and Gd-PPNP was attributed to the dense chains of PVA distributed within the shell, which creates a diffusion barrier such that the Gd is concealed in the core. In contrast, Gd-FFNP displayed a fast release of gadodiamide due to unstable structure, and the cumulative amount of gadodiamide released therefrom within 5 hours was greater than 70%.

### 4. Gd³⁺ Release In Vivo

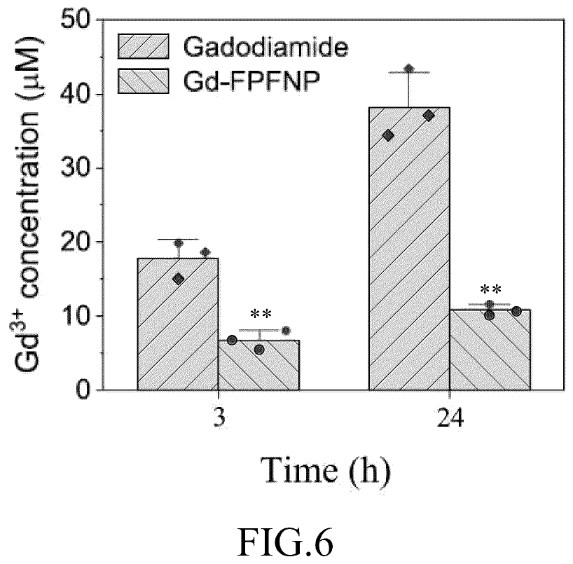

In order to monitor the in vitro release of gadolinium(III) ions (Gd³⁺) from gadodiamide and Gd-FPFNP, a respective one of gadodiamide at concentration of 1.05 mM and Gd-FPFNP that was loaded with 1.05 mM of gadodiamide were incubated in DMEM, and then added into a dialysis tube with cellulose membrane having a molecular weight cutoff of 1 kDa, followed by immersing the dialysis tube in 10 mL of PBS and stirring at 37° C. Thereafter, 0.5 mL aliquots of the PBS were taken out at the 3^{rd} hour and 24^{th} hour of stirring, followed by addition of Xylenol orange agent and analysis using a UV-Vis detector conducted according to procedures described by Barge A. et al., in an article entitled “How to determine free Gd and free ligand in solution of Gd chelates. A technical note.” published in Contrast Media Mol. Imaging, 2006, Vol. 1, p. 184-188, so as to determine the amount of Gd³⁺ released in vitro. This experiment was conducted in triplicates, and the results are shown in FIG. 6.

FIG. 6 shows concentrations of Gd³⁺ released from gadodiamide and Gd-FPFNP at different times. As shown in FIG. 6, at the 24^{th} hour, the Gd³⁺ detected after release from gadodiamide had a concentration of 38.6 μM, whereas the Gd³⁺ detected after release from Gd-FPFNP had a concentration of 10.8 μM, indicating that in comparison to gadodiamide, Gd-FPFNP are capable of effectively concealing gadodiamide in the core to prevent gadodiamide from leakage and dissociation into Gd³+.

### 5. Determination of Internalization (Cellular Uptake) Efficiency

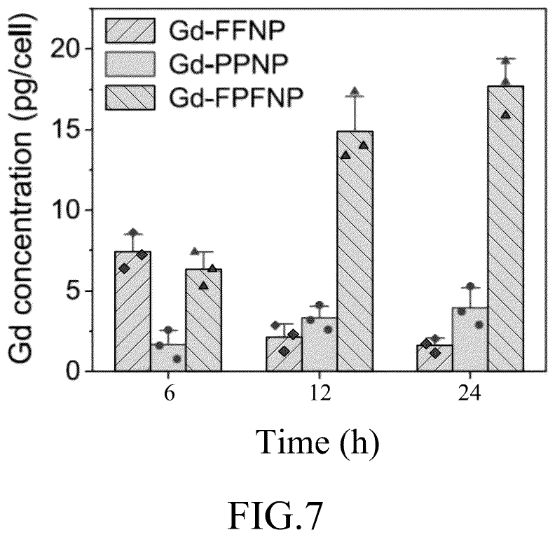

Since the therapeutic efficiency of gadolinium-neutron capture therapy (Gd-NCT) relies on the amount of gadodiamide accumulated at the GBM cells, the internalization efficiency of Gd-FFNP, Gd-PPNP and Gd-FPFNP was investigated at different time points. A respective one of the Gd-FFNP, Gd-PPNP and Gd-FPFNP was subjected to determination of internalization efficiency (i.e., cellular uptake efficiency) using UMSCs. In brief, the UMSCs were seeded at 1×10⁶ cells per well into respective wells of 6-well plates containing serum-free DMEM medium, and then treated by incubation with a respective one of the Gd-FFNP, Gd-PPNP and Gd-FPFNP for a time periods of 6 hours, 12 hours and 24 hours, with a magnet applied under the 6-well plates for 6 hours, i.e., the Gd-FFNP, Gd-PPNP and Gd-FPFNP were subjected to magnetic navigation to enhance accumulation thereof in the UMSCs. Thereafter, the resultant Gd-FFNP-treated UMSCs, Gd-PPNP-treated UMSCs, and Gd-FPFNP-treated UMSCs collected at the 6^{th} hour, 12^{th} hour and 24^{th} hour of incubation were calculated to determine numbers thereof, followed by hydrolysis using concentrated nitric acid (Fisher Scientific) at 80° C. for 60 minutes. The amounts of gadodiamide in the hydrolyzed Gd-FFNP-treated UMSCs, Gd-PPNP-treated UMSCs, and Gd-FPFNP-treated UMSCs were quantified using Agilent 7800 inductively coupled plasma mass spectrometry (ICP-MS) system. The ICP-MS system has an analytical range for elemental gadolinium that was set from 0.1 ng/mL to 1000 ng/mL, and was performed under the following analytical parameters: (i) peripump/integrated sample introduction system (ISIS) settings include, at the pre-run mode, uptake speed of 0.5 rps, uptake time of 30 seconds, and stabilization time of 20 seconds, and at the post-run mode, rinse speed of 0.5 rps, rinse at sample rinse port of 10 seconds, and rinse at standard rinse port of 10 seconds; (ii) plasma settings include radiofrequency (RF) power of 1550 W, RF matching of 1.70 V, sample depth of 10.0 mm, nebulizer gas of 1.05 L/min, nebulizer pump of 0.10 rps, S/C temperature of 2° C.; and (iii) ion lenses conditions include extract 1 of 0 V, extract 2 of −200 V, omega bias of −80 V, omega lens of 7.6 V, cell entrance of −40 V, cell exit of −60 V, deflect of 0.6 V, and plate bias of −55 V. This experiment was conducted in quadruplicates, and the results are shown in FIG. 7.

FIG. 7 shows concentration of gadodiamide in Gd-FFNP-treated UMSCs, Gd-PPNP-treated UMSCs, and Gd-FPFNP-treated UMSCs at different times. As shown in FIG. 7, at the 6^{th} hour after start of incubation, the concentration of gadodiamide in Gd-FFNP-treated UMSCs were higher compared with those of Gd-PPNP-treated UMSCs and Gd-FPFNP-treated UMSCs; however, the concentration of gadodiamide in Gd-FFNP-treated UMSCs significantly decreased at the 12^{th} and 24^{th} hours after start of incubation, indicating that Gd-FFNP had a low ability to retain gadodiamide therein, which was consistent with the fast release of gadodiamide from the Gd-FFNP as shown in FIG. 5. In contrast, the concentration of gadodiamide in Gd-FPFNP-treated UMSCs steadily increased from the 6^{th} hour to 24^{th} hour after start of incubation, and the Gd-FPFNP-treated UMSCs had concentration of gadodiamide that is higher compared with those of the Gd-PPNP-treated UMSCs and Gd-FFNP-treated UMSCs at the 24^{th} hour after start of incubation. The substantially enhanced gadodiamide concentration in the Gd-FPFNP-treated UMSCs was attributed to the slow release of gadodiamide and the enhanced cellular uptake promoted by the presence of fucoidan on the surface (i.e., outer shell layer) of Gd-FPFNP.

### Example 2. Evaluation of Internalization of Gadodiamide-Loaded Magnetic Nanoparticles (Gd-FPFNP) into Umbilical Cord-Derived Mesenchymal Stem Cells (UMSCs)

Since UMSCs treated with the Gd-FPFNP of the present disclosure had a relatively high amount of gadodiamide loaded therein and demonstrated slow release of gadodiamide, the internalization of Gd-FPFNP into UMSCs was further evaluated in this example so as to determine the optimal conditions for preparing the drug delivery system.

### A. Electron Microscopy and Flow Cytometry Analysis

The UMSCs were seeded at 1×10⁵ cells per well into respective wells of 6-well plates containing serum-free DMEM medium, and then treated by incubation with Gd-FPFNP at iron concentration of 100 μg Fe/mL for 6 hours, 12 hours, 24 hours, 48 hours or 5 days, in which a magnet applied under the 6-well plates for 6 hours. Thereafter, the serum-free medium was removed, followed by washing with PBS (pH 7.4) 3 times to ensure complete removal of the Gd-FPFNP and gadodiamide.

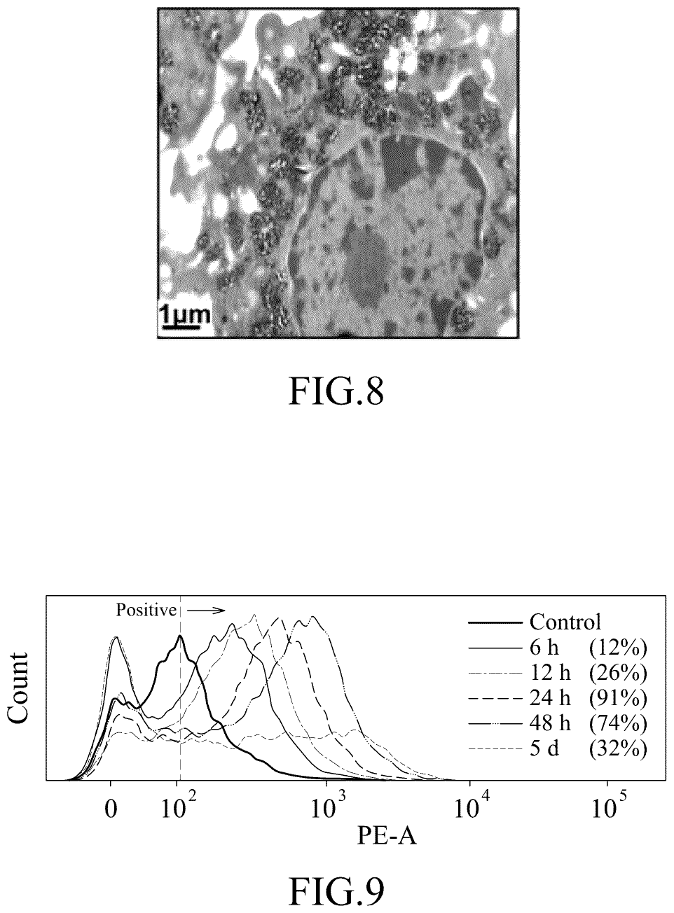

At the 24^{th} hour after start of incubation, the resultant Gd-FPFNP-treated UMSCs (i.e., the drug delivery system) were subjected to imaging so as to determine the subcellular localization of Gd-FPFNP in the UMSCs. In brief, the drug delivery system was fixed with glutaraldehyde (2.5%) in sodium cacodylate (0.05 M, pH 7.4) for 40 minutes and then embedded in agarose (2%), followed by staining with osmium tetroxide (2%) and uranyl acetate (0.5%) and further processing for ultrathin sectioning. The thus processed drug delivery system was subjected to imaging and photography using a transmission electron microscope (Manufacturer: Philips; Model no.: CM-120) at an acceleration voltage of 80 kV. The result is shown in FIG. 8.

FIG. 8 is a transmission electron microscopy image of Gd-FPFNP-treated UMSCs (i.e., the drug delivery system). As shown in FIG. 8, the internalized Gd-FPFNP accumulated in the cytoplasm of the UMSCs at the 24^{th} hour after start of incubation.

The efficiency of cellular uptake of Gd-FPFNP into UMSCs at different times (i.e., at the 6^{th} hour, 12^{th} hour, 24^{th} hour, 48^{th} hour and 5 days) of incubation were analyzed using BD FACSCalibur™ flow cytometer (Manufacturer: BD Biosciences). Gd-FPFNP were labeled with quantum dots according to the manufacturer's instruction to facilitate observation, while UMSCs not subjected to treatment with the Gd-FPFNP were prepared as control. The result is shown in FIG. 9.

FIG. 9 is a flow cytometry diagram illustrating the efficiency of cellular uptake of Gd-FPFNP into UMSCs at different times. As shown in FIG. 9, the fluorescence intensity of the UMSCs reached a plateau between the 12^{th} hour and 24^{th} hour after start of incubation and then decreased after the 48^{th} hour after start of incubation, which was most likely due to cell division and partial exocytosis, while at the 24^{th} hour after start of incubation, the Gd-FPFNP-treated UMSCs showed a fluorescence intensity that is 91% stronger compared with that of the non-treated UMSCs (control), suggesting that in preparation of the drug delivery system, the optimal time period for incubating UMSCs and Gd-FPFNP was between 12 hours and 24 hours.

### B. Cell Viability Analysis

Since Gd³⁺ released from gadodiamide may adversely affect the viability of the drug delivery system during transport thereof to the GBM cells, cytotoxicity assay were conducted after internalization of Gd-FPFNP into the UMSCs. The viability of the drug delivery system were determined by MTT assay conducted according to the manufacturer's protocol (Sigma Aldrich). In brief, UMSCs were seeded at 1×10⁵ cells per well into respective wells of 6-well plates containing serum-free DMEM medium, and then treated by incubation with Gd-FPFNP at iron concentrations of 1, 10, 50, 100 and 150 μg/mL, respectively. At the 24^{th} hour of incubation, the serum-free medium was replaced with a fresh serum-free medium containing 0.8 mg/mL of MTT dye. The resultant reaction solution was further incubated for 4 hours and then subjected to light absorbance measurement at a wavelength of 560 nm (OD₅₆₀) using a 96-well SpectraMax 190 microplate reader (Manufacturer: Molecular Devices).

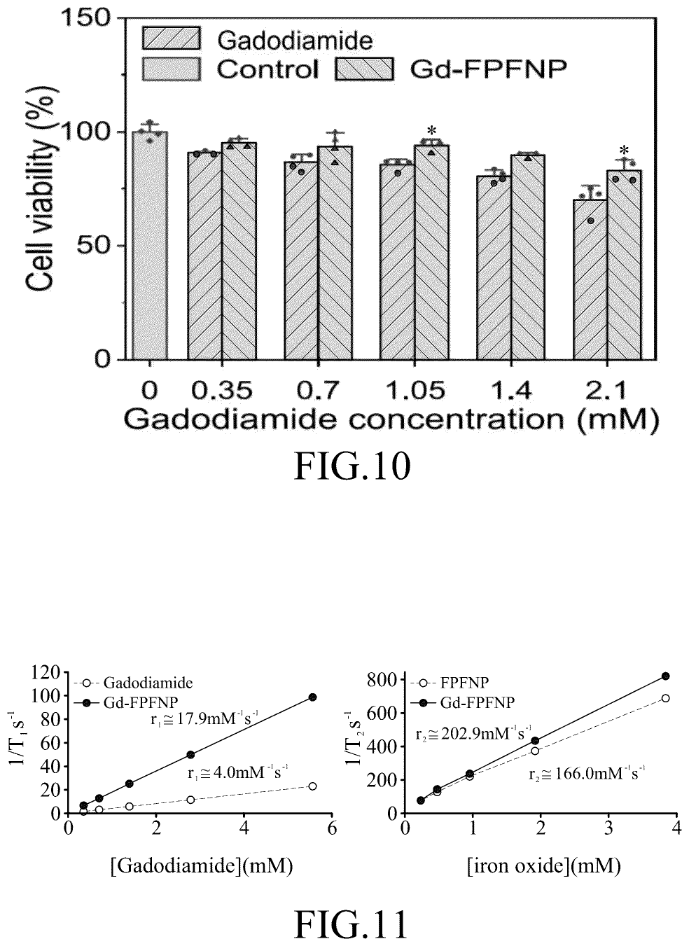

For comparison, UMSCs were subjected to treatment by incubation with gadodiamide (i.e., gadodiamide which was not subjected to the emulsification reactions as mentioned in Section A of Example 1, and thus was not loaded into the SPIO nanoparticles) at concentrations of 0.35, 0.7, 1.05, 1.4, and 2.1 mM, respectively, and then processed and subjected to the MTT assay as mentioned above. It should be noted that, in this experiment, Gd-FPFNP with iron concentrations of 1, 10, 50, 100 and 150 μg/mL were deemed equivalent to have gadodiamide at concentrations of 0.35, 0.7, 1.05, 1.4, and 2.1 mM, respectively. In addition, UMSCs which were not subjected to treatment with gadodiamide or Gd-FPFNP served as a control. This experiment was conducted in quadruplicates, and the results are shown in FIG. 10.

FIG. 10 shows the cell viability of Gd-FPFNP-treated UMSCs and gadodiamide-treated UMSCs. As shown in FIG. 10, at the 24^{th} hour after start of incubation, Gd-FPFNP-treated UMSCs had a cell viability that was higher compared with that of gadodiamide-treated UMSCs at the equivalent gadodiamide concentrations of 0.35, 0.7, 1.05, 1.4 and 2.1 mM. Since UMSCs treated with Gd-FPFNP loaded with gadodiamide at concentration of 1.05 mM for 24 hours showed excellent cell viability that was similar to that of the non-treated UMSCs (i.e., control), Gd-FPFNP loaded with 1.05 mM of gadodiamide are expected to have a high biocompatibility with the UMSCs.

C. Real-Time Tracking with Magnetic Resonance Imaging (MRI)

Since gadodiamide and iron oxide are both MRI contrast agents, the drug delivery system can be tracked in vivo with a dual-imaging strategy. In brief, gadodiamide and Gd-FPFNP each at concentrations ranging from 0.1 mM to 5.0 mM were prepared for measurement of T1 (spin-lattice relaxation time) MR relaxation time, while FPFNP and Gd-FPPNP each at concentrations ranging from 0.1 mM to 5.0 mM were prepared for measurement of T2 (spin-spin relaxation time) MR relaxation time. The T1 and T2 relaxation times were measured using a relaxometer (0.5 Tesla, 20 MHz, 37° C.), and the relaxation rates (1/T₁s⁻¹ and 1/T₂^{s-1}) were measured and plotted against the concentrations of gadodiamide or iron oxide. The relaxivities, r₁, and r₂, were respectively calculated from the slopes of curves.

FIG. 11 shows (a) the MR relaxation rates of gadodiamide and Gd-FPFNP at different concentrations of gadodiamide and (b) the MR relaxation rates of FPFNP and Gd-FPFNP at different concentrations of iron oxide. As shown in FIG. 11 (a), the r₁ values of gadodiamide and Gd-FPFNP were 4.0 mMs⁻¹ and 17.9 mMs⁻¹, respectively, and the r₁ value of Gd-FPFNP increased significantly compared with that of gadodiamide. As shown in FIG. 11(b), the r₂ value value of FPFNP (i.e., 166.0 mM-1s-1) was slightly lower than that of Gd-FPFNP (i.e., 202.9 mM-1s-1), indicating that the interference effect of gadodiamide on the r₂ value was minor. These results suggest that Gd-FPFNP could qualify as a MRI contrast agent due to its high r₁ value and low r₂/r₁ ratio.

In addition, gadodiamide-treated UMSCs and the drug delivery system were subjected to observation using MRI. In brief, UMSCs were seeded at 1×10⁵ cells per well into respective wells of 6-well plates containing serum-free DMEM medium, and then treated by incubation with gadodiamide at concentrations of 0.01, 0.1, 1.0 and 10.0 μg/mL, respectively, or with Gd-FPFNP loaded with gadodiamide at concentrations of 0.01, 0.1, 1.0 and 10.0 μg/mL, respectively, followed by dispersing in 2% agarose. Thereafter, MRI was conducted using a 7-Tesla (7-T) PharmaScan (Bruker). To be specific, T1-weighted images were obtained by T1-FLASH sequences with the parameters set as follows: TE=9 ms, TR=500 ms, matrix size=256×256, and NEX=16. T2-weighted images were obtained by T2 Rapid Acquisition with Relaxation Enhancement (RARE) sequences with the parameters set as follows: TE=19 ms, TR=3000 ms, matrix size=256×256, and NEX=1.

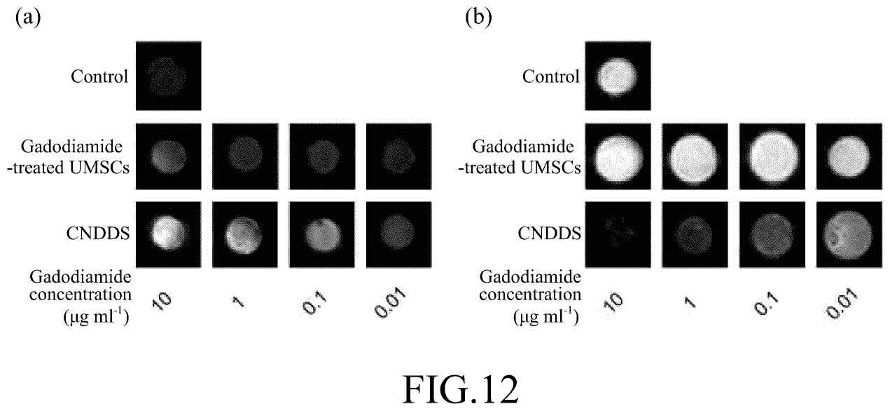

FIG. 12 shows (a) the T1-weighted images (T1WI) of gadodiamide-treated UMSCs (U-Gd) and the drug delivery system (DDS), and (b) the T2-weighted images (T2WI) of U-Gd and DDS. As shown in FIGS. 12 (a) and (b), the drug delivery system demonstrated intensity on both T1WI and T2WI which was significantly stronger than that of gadodiamide-treated UMSCs, indicating that association of the drug delivery system with the Gd-FPFNP was much more effective than that with the gadodiamide.

### Example 3. Evaluation of Biodistribution of Cell-Nanoparticle Drug Delivery System and Efficiency Thereof in Inhibiting Growth of Glioblastoma Multiforme (GBM) Cells

In order to further investigate the biodistribution of the cell-nanoparticle drug delivery system and efficiency of the same in inhibiting growth of GBM cells, the following experiments were conducted.

### A. Preparation of Cell-Nanoparticle Drug Delivery System

UMSCs were treated with Gd-FPFNP loaded with 1.05 mM of gadodiamide for 24 hours as described in section B of Example 2, and the resultant cell-nanoparticle drug delivery system was subjected to magnetic purification to remove non-internalized Gd-FPFNP.

### B. Preparation of Orthotopic GBM-Bearing Rats

Orthotopic GBM-bearing rats were prepared by implanting the female F344/NNral rats as described in the section entitled “4. Experimental animals” of the General Experimental Materials with the F98-Luc cells as described in the section entitled “2. Source and cultivation of glioblastoma multiforme (GBM) cells” of the General Experimental Materials via stereotaxic implantation according to procedures described by Towner R. A. et al., in an article entitled “Regression of glioma tumor growth in F98 and U87 rat glioma models by the Nitrone OKN-007” published in Neuro Oncol., 2013, Vol. 15, p. 330-340. In brief, female F344/NNral rats (8 weeks old) were anesthetized by intraperitoneal injection of chloral hydrate (Sigma Aldrich), and the thus anesthetized rats were then placed in a stereotactic frame, followed by drilling the skull of the anesthetized rats using a micro-burr to expose the brain. Thereafter, the F98-Luc cells, at an amount of 1×10⁵ cells in a volume of 2 μL, were introduced by intracerebral injection into the right striatum of the brain using a Hamilton syringe having a 26-gauge needle and a capacity of 10 μL. The intracerebral injection was conducted at stereotaxic coordinates as follows: AP=1; L=2.5; and V=4. After the F98-Luc cells were completely injected, the syringe was held in place for additional 5 minutes, followed by sealing the hole on the skull using bone wax, thereby obtaining the orthotopic GBM-bearing rats which were subjected to the following experiments.

### C. Tumor-Homing Ability

UMSCs have been reported to possess tumor-homing activity. To be specific, the CXCR4 and CCR2 expressed on UMSCs can interact with the overexpressed SDF-1α and MCP-1 in GBM, so as to provide tumor-homing tropism. In addition, after activation by SDF-1α, VLA-4 on the surface of UMSCs can interact with VCAM-1 adhesion molecules and β1 integrin on endothelial cells of the blood-brain barrier (BBB) and aid penetration of the BBB by rolling and migration to achieve intracranial delivery of gadolinium agent.

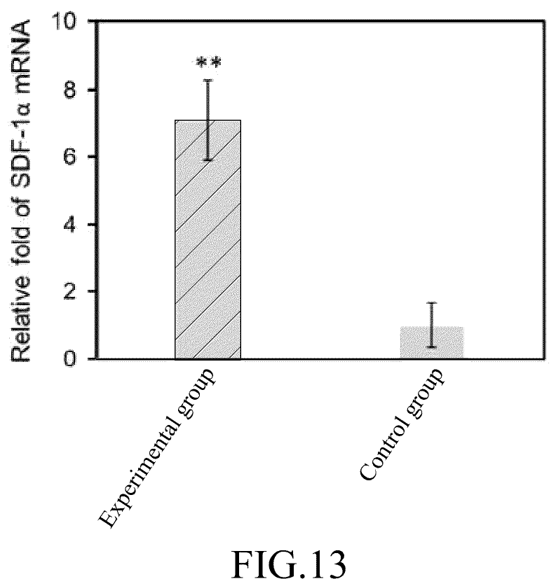

In brief, the orthotopic GBM-bearing rats (n=3) prepared in section B above served as experimental group while healthy female F344/NNral rats (8 weeks old; n=3), which were not implanted with the F98-Luc cells, served as control group. The rats in the experimental and control groups were subjected to determination of SDF-1α expression levels. In brief, total RNA was extracted from the brain of each rat by single-step isolation method using Trizol (Invitrogen), and then reverse-transcribed with random hexanucleotides using the SuperScript III First-Strand Synthesis System (Invitrogen) according to procedures described by Yang L. et al., in an article entitled “Size dependent distribution and toxicokinetics of iron oxide magnetic nanoparticles in mice” published in Nanoscale, 2015, Vol. 7, p. 625-636, followed by PCR with JumpStart Taq DNA polymerase (Sigma) using genomic engineering techniques known to those skilled in the art. Relative SDF-1a expression levels were determined by the critical threshold (Ct) number and calculated using the 2^{-ΔΔCt} method, with GADPH used as the reference gene. The results are shown in FIG. 13.

FIG. 13 shows the relative fold of SDF-1α mRNA levels in the brains of the rats in each group. As shown in FIG. 13, the SDF-1α mRNA levels in the brains of the rats in the experimental group were significantly higher than that in the control group, indicating that the abundance of SDF-1α in the GBM microenvironment may promote penetration of the BBB by the drug delivery system.

### D. Accumulation of Cell-Nanoparticle Drug Delivery System in GBM Cells

At 10 days post-implantation with the F98-Luc cells, the orthotopic GBM-bearing rats (n=6) prepared in section B above were administered with the cell-nanoparticle drug delivery system by intracarotid injection at a dose of 2×10⁶ cells. At the 12^{th}, 24^{th}, and 48^{th} hour post-administration of the cell-nanoparticle drug delivery system, the rats were placed on a thermostatically controlled heating pad to be gently warmed, and then subjected to T1-weighted magnetic resonance imaging (MRI) using SIGNA™ 3T MRI scanner (GE HealthCare) and bioluminescent imaging (BLI) using IVIS® Imaging System 200 Series (Caliper) so as to observe the accumulation of the cell-nanoparticle drug delivery system in the brain. For spin echo T1-weighted imaging (T1W1) of MRI, images were acquired with repetition time/echo time (TR/ET) of 500/15 ms, and field of view of 28×44 mm, and 22 coronal and axial images with 0.7 mm thick slices were captured for each rat. For BLI, at the 15th minute after the rats were administered with D-luciferin via intraperitoneal injection at a dose of 270 mg/kg, image acquisition was performed by determining region of interest (ROI) encompassing the intracranial area with bioluminescent signal using Living Image 3.0 software (Xenogen).

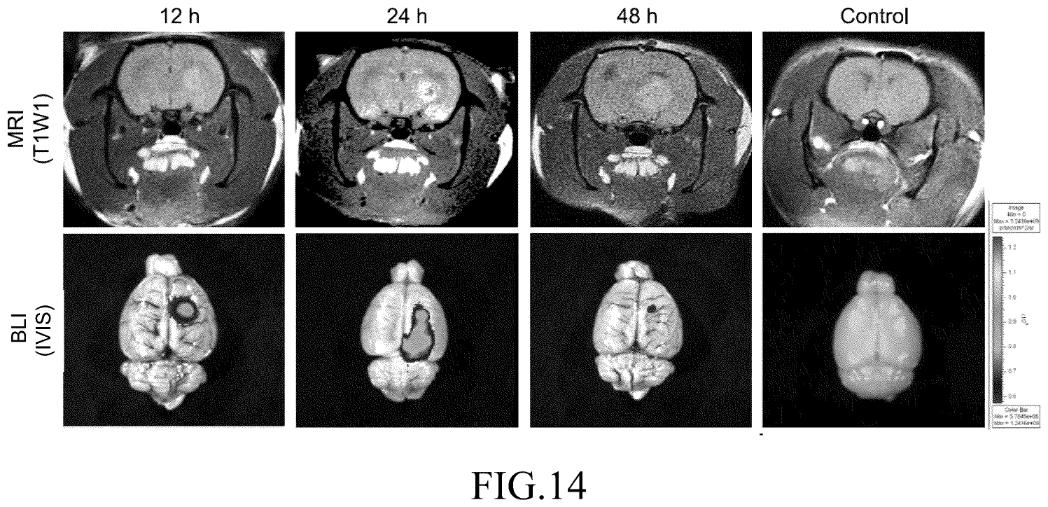

Healthy female F344/NNral rats (8 weeks old; n=6), which were not implanted with the F98-Luc cells, served as control. The results are shown in FIG. 14.

FIG. 14 shows MRI and BLI images of the brain of the orthotopic GBM-bearing rats at different times post administration of the cell-nanoparticle drug delivery system. As shown in FIG. 14, the signal in the T1WI images of MRI which indicated the presence of gadodiamide (released from the cell-nanoparticle drug delivery system) at the site of tumor peaked at the 24^{th} hour post-administration (see the upper lane), while the signal in the IVIS images of BLI which represented luciferase emitted from the F98-Luc cells also peaked at the 24th hour post-administration (see lower lane).

### E. Biodistribution of Cell-Nanoparticle Drug Delivery System in Vital Organs at Different Times

In order to accurately quantify the content of elemental gadolinium in vital organs of the orthotopic GBM-bearing rats at different times post-administration of cell-nanoparticle drug delivery system, the following experiment was conducted. The orthotopic GBM-bearing rats (n=3) were prepared as described in section B above, except that, in this experiment, after the skull of each of the anesthetized rats was drilled, the exposed brain was separated into right and left contralateral hemisphere, and then F98-Luc cells were only implanted into the right contralateral hemisphere (abbreviated as “right brain” hereinafter), whereas the left contralateral hemisphere (abbreviated as “left brain” hereinafter) was not implanted with the F98-Luc cells. At day 10 post-inoculation with the F98-Luc cells, the cell-nanoparticle drug delivery system was administered by intracarotid injection at a dose of 2×10⁶ cells to the orthotopic GBM-bearing rats. At the 12^{th} hour, 24^{th} hour, 48^{th} hour and 7 days post-administration, the orthotopic GBM-bearing rats administered with the cell-nanoparticle drug delivery system were sacrificed, and the blood and tissues from the heart, liver, spleen, lung, kidney, intestine, left brain and right brain were collected and then subjected to pre-processing steps including dessication at 90° C. for 4 to 6 hours, weighing and digestion using concentrated nitric acid (Fisher Scientific) at 90° C. for 60 minutes, followed by quantification of elemental gadolinium content by ICP-MS system as described in Item 4 of section B in Example 1. The results were compared with standard curve generated from internal standards (Geel, Belgium) and the National Institute of Standards and Technology (Gaithersburg, USA), and expressed as percentage of administered (injected) dose per gram tissue (ID/g), which was calculated using the following formula (3):

G=(H/I)×100% (3)
wherein G=administered dose per gram tissue (%)
   H=dose of cell-nanoparticle drug delivery system in tissue÷total dose of cell-nanoparticle drug delivery system administered
   I=weight of tissue

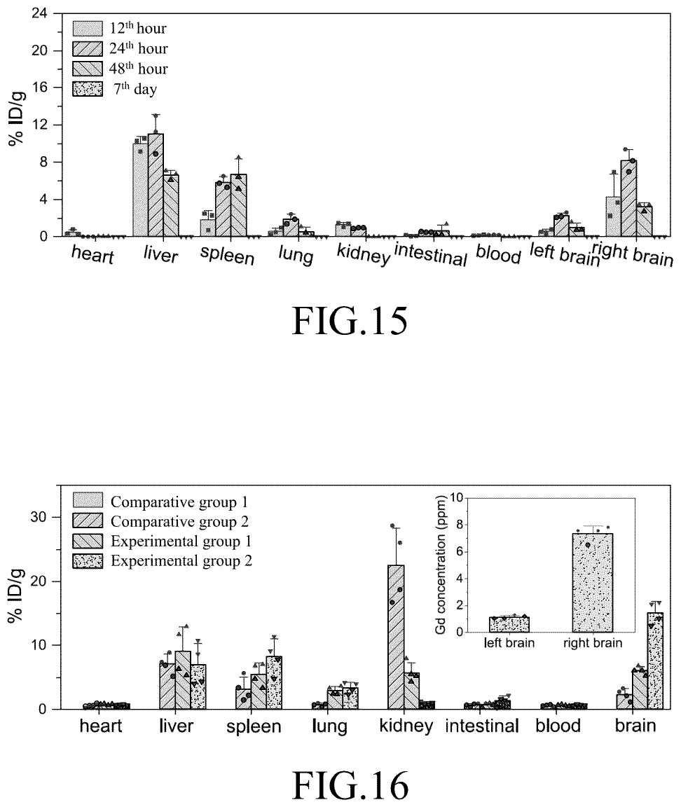

The results are shown in FIG. 15.

FIG. 15 shows the contents of elemental gadolinium in vital organs of the orthotopic GBM-bearing rats at different times post-administration of the cell-nanoparticle drug delivery system. As shown in FIG. 15, consistent with the results shown in FIG. 14, the content of elemental gadolinium in the right brain (where F98-Luc cells were implanted) reached a plateau at the 24^{th} hour post-administration of the cell-nanoparticle drug delivery system, suggesting that the ideal time point to perform neutron beam irradiation on the orthotopic GBM-bearing rats would be at the 24th hour post-administration of the cell-nanoparticle drug delivery system. In addition, the content of elemental gadolinium in the right brain was significantly higher than that in the left brain, indicating that the cell-nanoparticle drug delivery system exhibited a homing effect for GBM cells. Moreover, elemental gadolinium was also distributed mainly in the liver and spleen, but was cleared within 7 days post-administration, leaving less than 10 ppb of elemental gadolinium detected in all of the vital organs, indicating that elemental gadolinium would not permanently remained in vivo and cause toxicity.

F. Biodistribution of Cell-Nanoparticle Drug Delivery System in Vital Organs with Different Treatment Regimens

In order to determine the differences in biodistribution of the cell-nanoparticle drug delivery system with those of gadodiamide and Gd-FPFNP in vital organs, the orthotopic GBM-bearing rats prepared in section B above were divided into 4 groups, namely, comparative groups 1 and 2 (CG1 and CG2), and experimental groups 1 and 2 (EG1 and EG2), with n=4 per each group. The orthotopic GBM-bearing rats in the CG1 and CG2 were respectively administered with gadodiamide at a dose of 0.2 mg/kg and Gd-FPFNP at a dose of 10 mg/kg, the orthotopic GBM-bearing rats in the EG1 were administered with 2×10⁶ cells of the cell-nanoparticle drug delivery system, whereas the orthotopic GBM-bearing rats in the EG2 were administered with 2×10⁶ cells of the cell-nanoparticle drug delivery system and then a magnet (purchased from Tun-Hwa Electronic Material Co., Ltd. and having a diameter of 5 mm, a height of 0.5 mm, and a magnetic field of 0.5 T) was immediately stuck onto the skull on the top of the right brain of each rat, i.e., the cell-nanoparticle drug delivery system was subjected to magnetic navigation in order to be delivered to the GBM cells. At the 24^{th} hour post-administration, the orthotopic GBM-bearing rats in each group were sacrificed, and the blood and tissues from the heart, liver, spleen, lung, kidney, intestine, left brain and right brain were subjected to the pre-processing steps as described in section E above, and then subjected to quantification of elemental gadolinium (Gd) content by ICP-MS system as described in Item 5 of section B in Example 1. The results were expressed as percentage of administered (injected) dose per gram tissue (ID/g), calculated using the aforesaid formula (3), and shown in FIG. 16.

FIG. 16 shows the contents of elemental gadolinium in vital organs of the orthotopic GBM-bearing rats in each group. As shown in FIG. 16, elemental gadolinium was not or hardly detected in most of the vital organs of the orthotopic GBM-bearing rats in the CG1, indicating that gadodiamide had a fast clearance in vivo. In contrast, elemental gadolinium was detected in most of the vital organs (except for the heart and blood) of the orthotopic GBM-bearing rats in the CG2, EG1 and EG2, indicating that the Gd-FPFNP and the cell-nanoparticle drug delivery system were relatively stable and that the gadodiamide loaded therein was released slowly. In addition, in comparison to CG2, the contents of elemental gadolinium detected in the kidney of the orthotopic GBM-bearing rats in the EG1 and EG2 were significantly reduced while the contents of elemental gadolinium detected in the brain of the orthotopic GBM-bearing rats in the EG1 and EG2 were significantly increased, suggesting that the cell-nanoparticle drug delivery system of the present disclosure is capable of overcoming the limitations of conventional drug delivery system in penetrating the blood-brain barrier and achieving localization in the GBM cells. Moreover, the content of elemental gadolinium detected in the brain of the orthotopic GBM-bearing rats in the EG2 (15.4% ID/g) was significantly higher than that in the EG1 (6.07% ID/g), indicating that the cell-nanoparticle drug delivery system administered under magnetic navigation enables a higher content of gadolinium to be accumulated in the GBM cells.

G. Tumor-to-blood (T/B) ratio and tumor-to-normal tissue (T/N) ratio

In order to determine whether the cell-nanoparticle drug delivery system showed preferential accumulation at the GBM cells and to evaluate the potential risk of the cell-nanoparticle drug delivery system to adjacent tissues of the brain, tumor-to-blood (T/B) ratio and tumor-to-normal tissues (T/N) ratio of the orthotopic GBM-bearing rats in each group were respectively calculated by substituting the thus obtained elemental gadolinium contents in the brain and blood as shown in section E of this example into the following formulas (4) and (5):

J=(K/L)×100% (4)
wherein J=T/B ratio
   K=concentration of elemental gadolinium in whole brain
   L=concentration of elemental gadolinium in blood

M=(N/O)×100% (5)
wherein M=T/N ratio
   N=concentration of elemental gadolinium in right brain
   O=concentration of elemental gadolinium in left brain

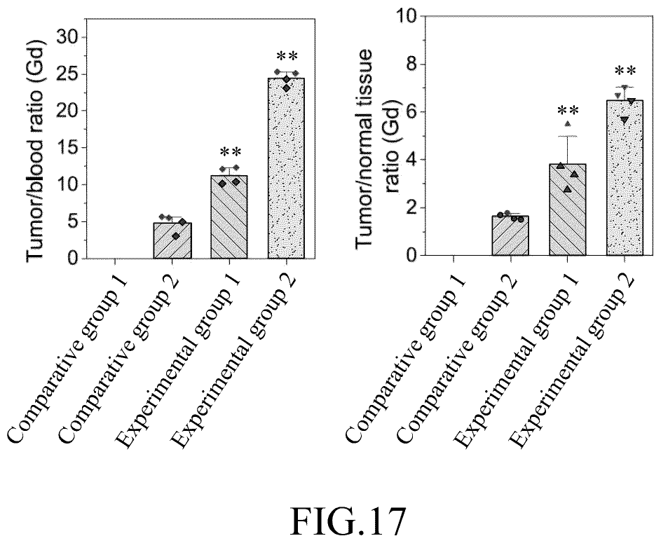

The results are shown in FIGS. 17(a) and (b).

FIGS. 17(a) and (b) respectively show the T/B ratio and the T/N ratio of the orthotopic GBM-bearing rats in each group. As shown in FIG. 17(a), the T/B ratio of the orthotopic GBM-bearing rats in the EG1 was 11.05, which was 2.32-fold higher than that in the CG2, while the orthotopic GBM-bearing rats in the EG2 showed a T/B ratio of 24.4, which was 2.22-fold and 5.19-fold higher than those of EG1 and CG2, respectively.

As shown in FIG. 17(b), the T/N ratios of the orthotopic GBM-bearing rats in the EG1 and EG2 were 3.83 and 6.46, respectively, which were 2.1-fold and 3.5-fold higher than that in the CG2. These results demonstrated that the cell-nanoparticle drug delivery system showed preferential accumulation at the GBM cells, which reduced potential risks to adjacent tissues of the brain, and that the cell-nanoparticle drug delivery system administered under magnetic navigation can effectively enhance the delivery of the same to the GBM cells, i.e., promoting localization effect.

### H. Interaction Between GBM Cells and UMSCs

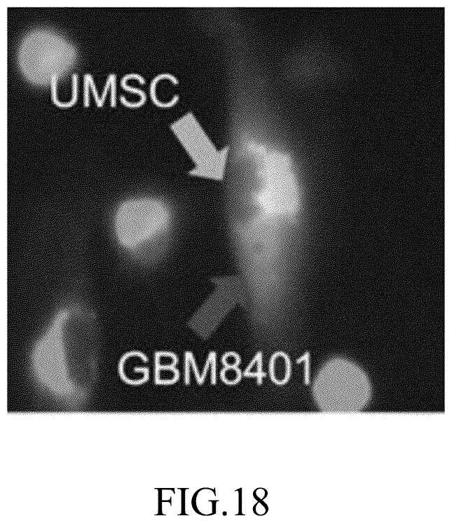

In order to investigate the interaction between GBM cells and UMSCs of the cell-nanoparticle drug delivery system mimicking in vivo environment, GBM8401 cells were co-cultivated with the cell-nanoparticle drug delivery system. First, GBM8401 cells were transfected with pDSRed-N1 (Clontech) and cultivated in DMEM medium supplemented with fetal bovine serum (10%), 100 U/mL of penicillin, and 100 mg/mL streptomycin at culture conditions set at 37° C. and 5% CO₂ so as to obtain GBM8401-RFP transformants expressing red fluorescent protein (RFP). In addition, UMSCs were transformed with Lenti-GFP vector using technology well known to those skilled in the art, and the resultant green fluorescent protein-expressing UMSCs were incubated with 100 μM of Gd-FPFNP for 12 hours so as to obtain Gd-FPFNP-treated GFP-expressing UMSCs, i.e., a cell nanoparticle-drug delivery system capable of expressing GFP (abbreviated as CNDDS-GFP). Next, the GBM8401-RFP was co-cultivated with the CNDDS-GFP in a cell number ratio of 10:1 under magnetic navigation for 24 hours, followed by colocalization imaging of the resultant fusion progeny using a confocal laser scanning microscope (Carl Zeiss LSM 510) at a magnification of 63 (objective lens)*10 (eyepiece) times. The result is shown in FIG. 18.

FIG. 18 shows a confocal laser scanning microscopy image of the fusion progeny observed after GBM8401-RFP was co-cultivated with the CNDDS-GFP for 24 hours. As shown in FIG. 18, the fusion progeny resulted from fusion of the UMSC of the CNDDS-GFP with the GBM8401 cell, in which the nucleus of the UMSC co-localized with the nucleus of the GBM8401 (see the arrows).

I. In Vitro Therapeutic Effect of Cell-Nanoparticle Drug Delivery System after Gadolinium-Neutron Capture Therapy

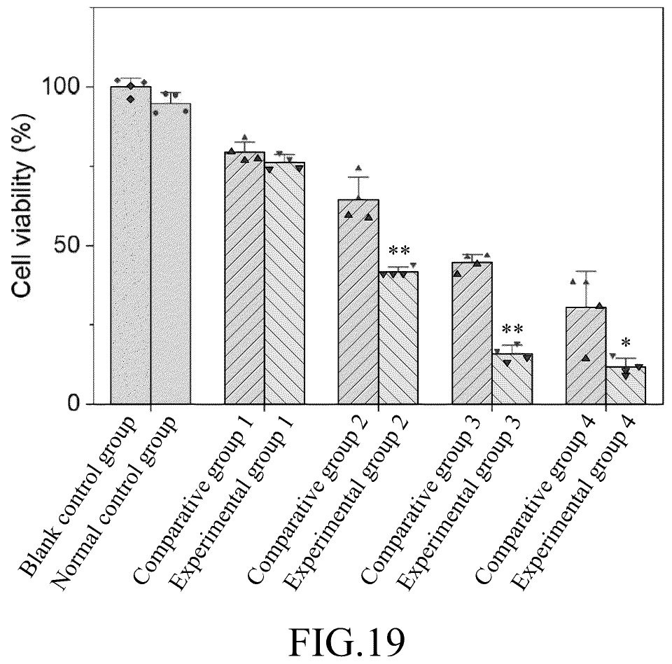

In order to compare the in vitro therapeutic effect of the cell-nanoparticle drug delivery system with that of gadodiamide on cell viability after gadolinium-neutron capture therapy, the following experiment was conducted. First, GBM8401 cells were divided into 10 groups, namely, a blank control group (BCG), a normal control group (NCG), 4 comparative groups, i.e., comparative groups 1 to 4 (CG1 to CG4) and 4 experimental groups, i.e., experimental groups 1 to 4 (EG1 to EG4), with the number of cells in each group being 1×10⁶. Next, the GBM8401 cells of the CG1 to CG4 were co-cultivated for 24 hours, under magnetic navigation, with gadodiamide at concentrations of 35, 175, 525 and 1050 μM, respectively, while the GBM8401 cells of the EG1 to EG4 were co-cultivated for 24 hours, under magnetic navigation, with the drug delivery system including Gd-FPFNP prepared by loading gadodiamide at concentrations of 35, 175, 525 and 1050 μM, respectively. The GBM8401 cells of the BCG and NCG were left to stand for 24 hours (i.e., not subjected to co-cultivation with gadodiamide or Gd-FPFNP). Thereafter, the GBM8401 cells of the NCG, CG1 to CG4, and EG1 to EG4 were subjected to gadolinium-neutron capture therapy by irradiation with a thermal neutron beam for 1 hour and 36 minutes at a rate of 2×10¹³ neutron/cm² using the Tsing Hua Open Pool Reactor, National Tsing Hua University, Taiwan, followed by observation for 24 hours. The cells of the BCG received no thermal neutron beam irradiation. Afterwards, cell viability analysis as described in section B of Example 2 were performed to determine the cell killing (i.e., tumoricidal) effect of the cell-nanoparticle drug delivery system and gadodiamide. This experiment were performed in quadruplicates, and the results are shown in FIG. 19.

FIG. 19 shows the cell viability of GBM8401 cells in each group at the 24^{th} hour post-irradiation with thermal neutron beam. As shown in FIG. 19, the cell viability of GBM8401 cells in the EG2, EG3 and EG4 was significantly lower than that of the CG2, CG3 and CG4, respectively, demonstrating that in comparison with gadodiamide, the cell-nanoparticle drug delivery system had enhanced tumoricidal effect on GBM8401 cells.

J. In Vivo Therapeutic Effect of Cell-Nanoparticle Drug Delivery System after Gadolinium-Neutron Capture Therapy

In order to determine the in vivo therapeutic effect of the cell-nanoparticle drug delivery system on the survival of orthotopic GBM-bearing rats after gadolinium-neutron capture therapy, the following experiment was conducted. First, orthotopic GBM-bearing rats were prepared as described in the section B of this example, except that the amount of F98-Luc cells inoculated by injection into the right striatum of each rat was 2×10⁶ F98-Luc cells, and then the rats were divided into 6 groups, namely, a blank control group (BCG), three comparative groups, i.e., comparative groups 1 to 3 (CG1 to CG3), and two experimental groups, i.e., experimental groups 1 and 2 (EG1 and EG2), with n=6 per each group. After 7 to 10 days post-inoculation, the rats in the BCG were administered with PBS (0.5 mL), the rats in the CG1 were administered with gadodiamide at a dose of 0.2 mg/kg, the rats in the CG2 and CG3 were administered with Gd-FPFNP (0.5 mL) at a dose of 10 mg/kg, while the rats in the EG1 and EG2 were administered with 2×10⁶ cells of the cell-nanoparticle drug delivery system. The administration of PBS, gadodiamide, Gd-FPFNP and the cell-nanoparticle drug delivery system to the rats in the respective group were conducted via intracarotid injection. In addition, the rats in the CG3 and EG2 were further subjected to magnetic navigation as described in section F of this example for 12 hours. At the 24^{th} hour post-administration, the rats in the CG1 to CG3 and EG1 and EG2 were subjection to gadolinium-neutron capture therapy by irradiation with a thermal neutron beam for 1 hour and 36 minutes at a rate of 2×10¹³ neutron/cm² using the Tsing Hua Open Pool Reactor, National Tsing Hua University, Taiwan. During the irradiation, the body of each rat was shielded with poly-(methyl-methacrylate) and polyethylene complex plastic plates so as to protect from undesirable radiation. The rats in the BCG received no thermal neutron beam irradiation.

It should be noted that, before the thermal neutron beam irradiation, the size of the tumor was measured using MRI, in which T2-weighted imaging (T2WI, TE: 50 ms; TR: 3000 ms; in-plane matrix size: 256×256; field of view: 2.56 cm) was conducted using rapid acquisition with relaxation enhancement (RARE) spin-echo sequence so as to determine boundaries of the tumor. The volume of the tumor was determined by compiling tumor areas from all slice images that contained tumors, was further quantified by ImageJ using the following formula (6):

P=Q×R (6)
wherein P=volume of tumor
   Q=tumor area
   R=slice thickness

At the 21^{st} day post-irradiation, the rats were sacrificed by anesthetizing by intraperitoneal injection with chloral hydrate (0.4 g/kg), and then subjected to transcranial perfusion with saline, followed by immersion in 4% paraformaldehyde. Thereafter, the tumor tissues were dehydrated in 30% sucrose, frozen on dry ice, and then cut in a series of adjacent coronal sections each having 6 μm thickness using a cryostat, followed by hematoxylin and eosin (H&E) staining and imaging using a light microscope (Nikon, Eclipse E600). Accurate measurement of the volume of tumor by a caliper at day 21 post-irradiation with thermal neutron beam was calculated using the following formula (7):

S=(T×U²)/2 (7)
wherein S=volume of tumor
   T=major axes of tumor sections
   U=minor axes of tumor sections

The median survival times of the rats in each group was determined using Kaplan-Meier survival curve with 95% confidence interval, whereas the survival distributions between the groups were determined by log-rank analysis.

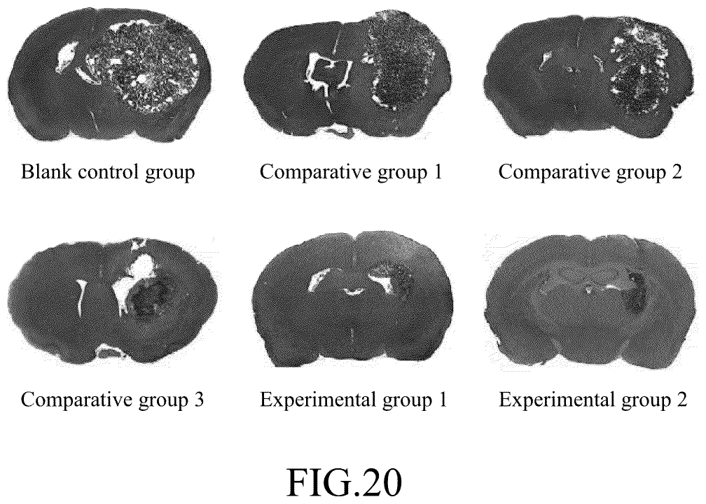

The results are shown in FIGS. 20 to 22.

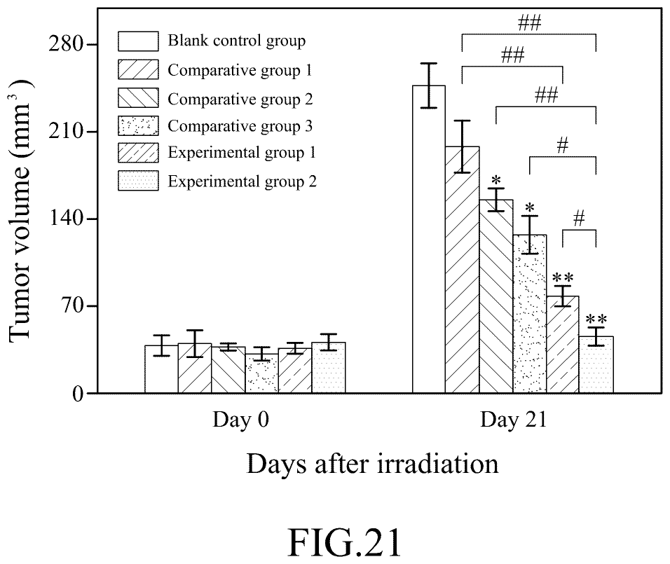

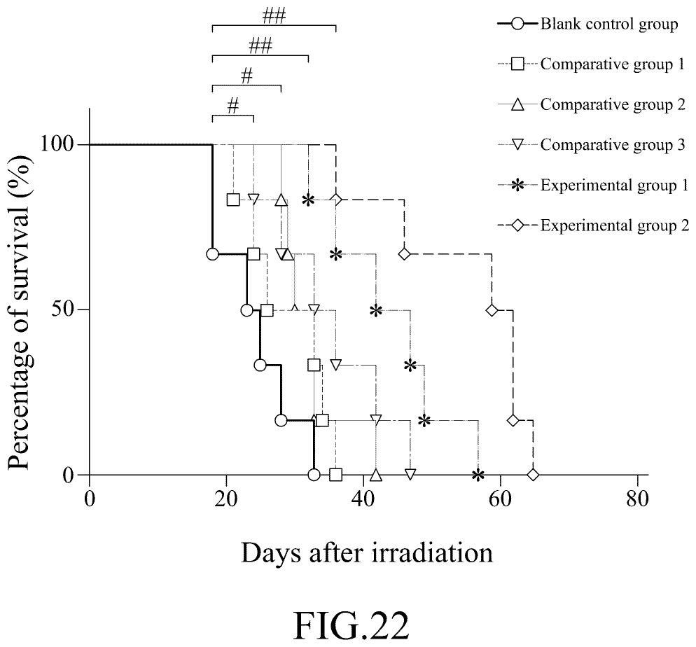

FIG. 20 shows light microscopy images of H&E staining of the brains of the orthotopic GBM-bearing rats in each group at day 21 post-irradiation with thermal neutron beam, FIG. 21 shows the tumor volumes of the orthotopic GBM-bearing rats in each group at day 0 and day 21 post-irradiation with thermal neutron beam, and FIG. 22 shows the percentage of survival for the orthotopic GBM-bearing rats in each group versus days post-irradiation with thermal neutron beam. As shown in FIGS. 20 and 21, in the BCG, the tumor had infiltrated through the entire right brain, invaded across the midline and grown to have a size approximately 5 times larger, suggesting the aggressive nature of GBM. As shown in FIGS. 20 to 22, the rats in the CG1 showed slight inhibition of tumor progression but fail to extend median survival time thereof when compared to those in the BCG, while the rats in the CG3 and EG2 showed significant inhibition of tumor invasion and progression compared to those in the CG2 and EG1, respectively, indicating that application of magnetic navigation promoted the therapeutic efficacy of the Gd-FPFNP and the cell-nanoparticle drug delivery system. In addition, the rats in the EG1 and EG2 showed significant inhibition of localized tumor invasion and significant reduction of tumor volume, as well as a significant increase in median survival times compared to those in the CG2 and CG3, indicating that the cell-nanoparticle drug delivery system showed therapeutic efficacy which was greater than that of Gd-FPFNP. These results showed that the cell-nanoparticle drug delivery system of the present disclosure, when administered under magnetic navigation, is capable of enhancing the therapeutic effect of gadolinium post-irradiation with thermal neutron beam, and thus, extends the survival time of the orthotopic GBM-bearing rats.

It should be noted that, the cell-nanoparticle drug delivery system of the present disclosure, which included the Gd-FPFNP loaded with 109.3 μg of gadodiamide therein, corresponded to a dose of 0.2 mg/kg in the orthotopic GBM-bearing rats when administered by intracarotid injection, and such dosage of gadodiamide was substantially lower than the amounts of gadolinium compounds used previously in neutron capture therapy for treatment of cancer. As shown in FIG. 20 to 22, the reduced dose of gadodiamide in the cell-nanoparticle drug delivery system administered to the orthotopic GBM-bearing rats did not compromise the therapeutic efficiency of the cell-nanoparticle drug delivery system in inhibition of GBM cells, but largely enhanced the safety profile of the cell-nanoparticle drug delivery system.

### K. Safety Assessment of Gd-FPFNP and Cell-Nanoparticle Drug Delivery System

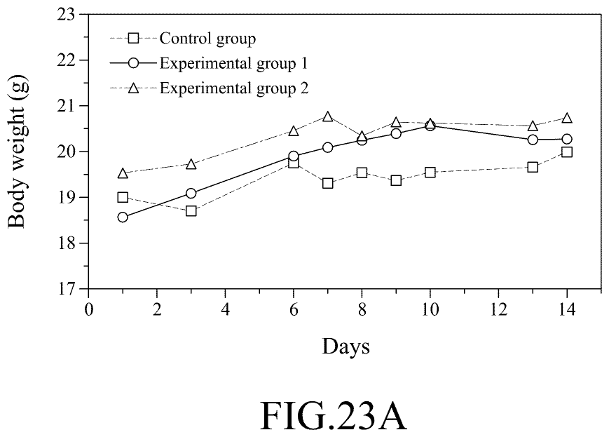

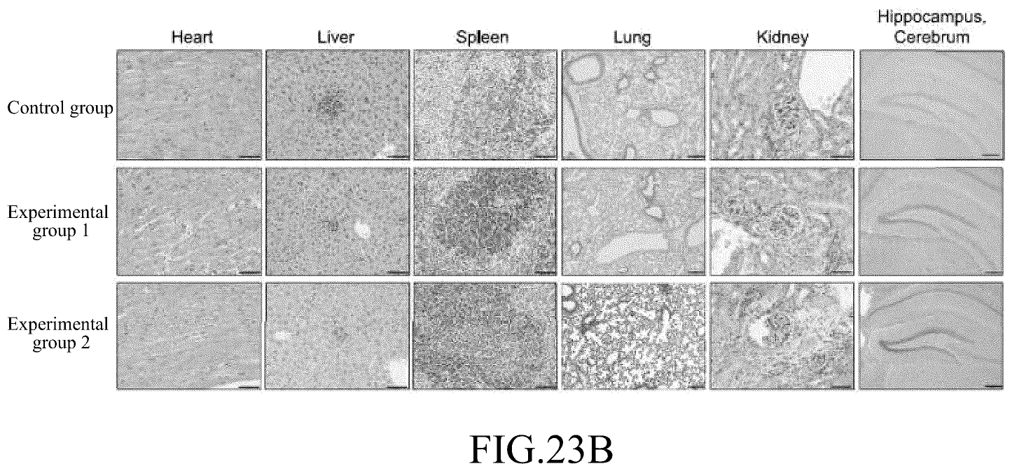

In order to determine the local and systemic safety profile of Gd-FPFNP and the cell-nanoparticle drug delivery system of the present disclosure, safety assessment was conducted using healthy mice. In brief, healthy 8-week old male C57BL/6 JNarl mice as described in Item 4 of the General Experimental Materials were divided into 3 groups, namely, control group, experimental group 1 (EG1), and experimental group 2 (EG2), with n=3 per each group. The mice in the control group, EG1 and EG2 were respectively administered, via intracarotid injection, with saline, Gd-FPFNP at a dose of 10 mg/kg, and 2×10⁶ cells of the cell-nanoparticle drug delivery system, followed by a 14-day clinical observation, in which mortality, body weight and clinical symptoms of the mice in each group were determined. Next, the mice in each group were sacrificed, and tissues from vital organs, i.e., heart, liver, lung, spleen, kidney and brain (hippocampus and cerebrum), were harvested and then preserved in 10% neutral buffered formalin at room temperature for 96 hours. Thereafter, the resultant tissue samples were sectioned by cutting using a microtome to obtain multiple tissue sections each having a thickness of 4 μm to 6 μm, followed by H&E staining and histological analyses. The results are shown in FIGS. 23A and 23B.

FIG. 23A shows body weight change of the healthy male C57BL/6 Narl mice in each group, while FIG. 23B shows representative H&E stain of vital organs of the healthy male C57BL/6 Narl mice in each group. As shown in FIG. 23A and FIG. 23B, the body weight of the mice in each group showed a similar trend without body weight loss over the 14-day observation period, while the histological analyses of the tissues of vital organs including heart, liver, spleen, lung, kidney and brain (hippocampus and cerebrum) at day 14 of the observation revealed that these tissues were within normal range and free of any abnormality related to gadodiamide or UMSC toxicity, such as neuron degeneration, inflammation, or lesions.

### Example 4. Evaluation of Anti-Inflammatory Effect of Gadodiamide-Loaded Magnetic Nanoparticles after Gadolinium-Neutron Capture Therapy

Fucoidan has been demonstrated to possess anti-inflammatory and neuroprotective effects. Since the gadodiamide-loaded magnetic nanoparticles, i.e., Gd-FPFNP, were coated with fucoidan, Gd-FPFNP may exert anti-inflammatory effect were administered to a subject diagnosed with GBM and then subjecting the subject to gadolinium-neutron capture therapy. In order to investigate whether Gd-FPFNP show a potential neuroprotective effect after gadolinium-neutron capture therapy, the following experiment was conducted.

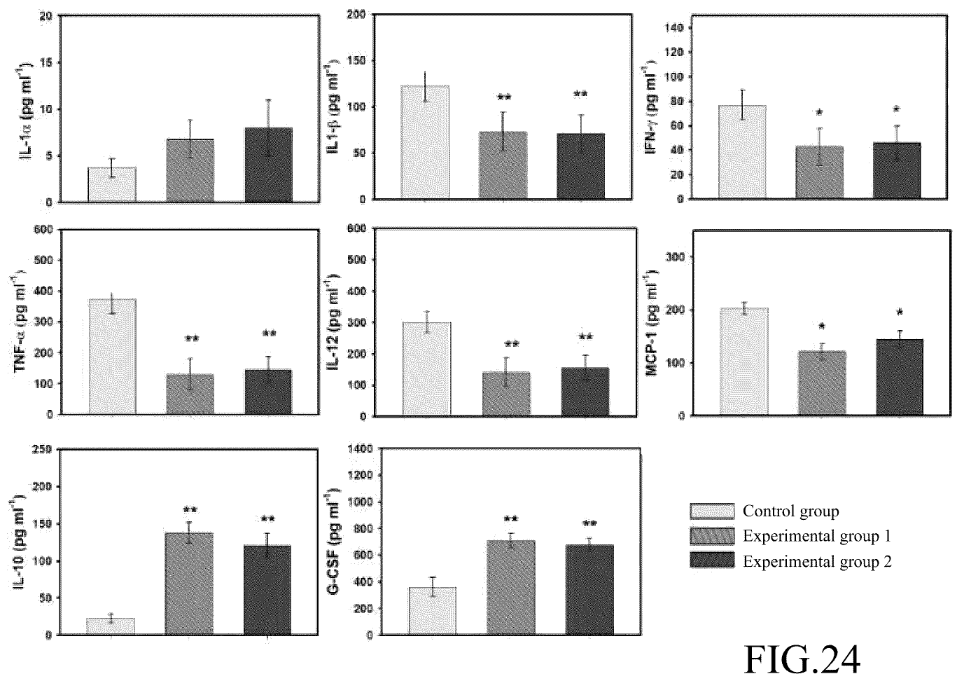

The orthotopic GBM-bearing rats were prepared as described in the section B of Example 3 were divided into 3 groups, namely, control group (CG), experimental group 1 (EG1), and experimental group 2 (EG2), with n=3 per each group. The rats in the control group were administered, via intracarotid injection, with saline, while the rats in the EG1 and EG2 were administered, via intracarotid injection, with Gd-FPFNP at a dose of 10 mg/kg. At the 24^{th} hour post-administration, blood was collected from the rats in each group and subjected to measurement of the serum levels of pro-inflammatory factors including IL-1α, IL-1β, IFN-γ, TNF-α, IL-12 and MCP-1, and anti-inflammatory cytokines including IL-10 and G-CSF using Bio-Plex cytokine reagent kit. In addition, the rats in the EG2 were further subjected to neutron beam irradiation as described in section J of Example 3. At the 24^{th} hour post-irradiation, blood were collected from the rats in each group and then subjected to measurement of the serum levels of the aforesaid pro-inflammatory factors and anti-inflammatory cytokines. Differences in the serum levels of the pro-inflammatory factors and anti-inflammatory cytokines measured at the 24^{th} hour post-administration and the 48^{th} hour post-administration (for CG and EG1) or 24^{th} hour post-irradiation (for EG2) for the rats in each group were calculated. The results are shown in FIG. 24.

FIG. 24 shows the differences in serum levels of pro-inflammatory factors and those of anti-inflammatory cytokines in the orthotopic GBM-bearing rats of each group. As shown in FIG. 24, the reduction in serum levels of IL-1β, IFN-γ, TNF-α, IL-12 and MCP-1 in the rats of EG1 and EG2 were greater compared with those of the control group, while the increase in serum levels of IL-10 and G-CSF in the rats of EG1 and EG2 were also greater compared to the control group, indicating that Gd-FPFNP induced a significant reduction in proinflammatory factors and a significant increase in anti-inflammatory cytokines, suggesting that Gd-FPFNP mediated immunomodulation-induced neuroprotection in orthotopic GBM-bearing rats to allow faster recovery of the brain tissues post-irradiation.

In summary, the aforesaid results suggest that the cell-nanoparticle drug delivery system of the present disclosure, which is prepared by loading a relatively low amount of gadolinium-based agent, i.e., gadodiamide, into iron oxide magnetic nanoparticles to obtain Gd-FPFNP which were then internalized into the UMSCs, overcomes the current limitations of gadolinium-neutron capture therapy, including off-target effects and rapid metabolism, and is capable of significantly inhibit the growth of GBM cells and extend median survival time of orthotopic GBM-bearing rats. Therefore, the cell-nanoparticle drug delivery system of the present disclosure is expected to have a high potential to be utilized for gadolinium-neutron capture therapy in treatment of cancer.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A cell-nanoparticle drug delivery system, comprising:
mesenchymal stem cells; and
gadolinium-based agent-loaded magnetic nanoparticles which are internalized into the mesenchymal stem cells, each of the gadolinium-based agent-loaded magnetic nanoparticles including a core and a shell, the core being loaded with gadolinium-based agent and including a fucoidan-based inner core layer with the fucoidan non-covalently bound to the gadolinium-based agent, the shell including a superparamagnetic iron oxide-based inner shell layer with the superparamagnetic iron oxide bound to the gadolinium-based agent through electrical attraction, and an outer shell layer made of fucoidan and polyvinyl alcohol.

2. The cell-nanoparticle drug delivery system as claimed in claim 1, wherein the gadolinium-based agent is gadodiamide.

3. The cell-nanoparticle drug delivery system as claimed in claim 1, wherein the fucoidan is obtained from a brown seaweed material selected from the group consisting of Cladosiphon okamuranus, Undaria pinnatifida, Laminaria japonica, and Fucus vesiculosus.

4. The cell-nanoparticle drug delivery system as claimed in claim 3, wherein the fucoidan has an average molecular weight ranging from 1 kDa to 200 kDa.

5. The cell-nanoparticle drug delivery system as claimed in claim 1, wherein based on a total weight of each of the gadolinium-based agent-loaded magnetic nanoparticles, the fucoidan is present in an amount ranging from 2 wt % to 60 wt %, the superparamagnetic iron oxide is present in an amount ranging from 0.15 wt % to 20.0 wt %, and the gadolinium-based agent is present in an amount ranging from 0.5 wt % to 40.0 wt %.

6. The cell-nanoparticle drug delivery system as claimed in claim 1, wherein each of the gadolinium-based agent-loaded magnetic nanoparticles has a particle size ranging from 50 nm to 500 nm.

7. The cell-nanoparticle drug delivery system as claimed in claim 1, wherein the mesenchymal stem cells are selected from the group consisting of umbilical cord-derived mesenchymal stem cells, adipose-derived mesenchymal stem cells, bone marrow-derived mesenchymal stem cells, and placenta-derived mesenchymal stem cells.

8. The cell-nanoparticle drug delivery system as claimed in claim 7, wherein the mesenchymal stem cells are umbilical cord-derived mesenchymal stem cells.

9. The cell-nanoparticle drug delivery system as claimed in claim 8, wherein the gadolinium-based agent is present in an amount ranging from 0.1 μg/cell to 30 μg/cell in the umbilical cord-derived mesenchymal stem cells.

10. The cell-nanoparticle drug delivery system as claimed in claim 1, wherein the gadolinium-based agent is present in an amount ranging from 0.1 μg/cell to 100 μg/cell in the mesenchymal stem cells.

11. A method for inhibiting the growth of tumor cells in a subject, comprising:
administering to the subject a cell-nanoparticle drug delivery system as claimed in claim 1 by injection;
navigating the cell-nanoparticle drug delivery system to the tumor cells of the subject using an external magnetic field; and
subjecting the tumor cells of the subject to neutron beam irradiation, so that gamma rays and internal convergent electrons emit from gadolinium-based agent to kill the tumor cells.

12. The method as claimed in claim 11, wherein the cell-nanoparticle drug delivery system is administered by one of intracarotid injection and intravenous injection.

13. The method as claimed in claim 12, wherein the cell-nanoparticle drug delivery system is administered by intracarotid injection.

14. The method as claimed in claim 11, wherein the tumor cells are selected from the group consisting of head and neck tumor cells, brain tumor cells, skin tumor cells, pancreatic tumor cells, liver tumor cells, and lung tumor cells.

15. The method as claimed in claim 14, wherein the brain tumor cells are glioblastoma multiforme cells.

16. A method for diagnosing tumor cells in a subject, comprising:
administering to the subject a cell-nanoparticle drug delivery system as claimed in claim 1 by injection;
navigating the cell-nanoparticle drug delivery system to the tumor cells of the subject using an external magnetic field; and
subjecting the subject to magnetic resonance imaging analysis so as to locate the tumor cells.

17. The method as claimed in claim 16, wherein the cell-nanoparticle drug delivery system is administered by one of intracarotid injection and intravenous injection.

18. The method as claimed in claim 17, wherein the cell-nanoparticle drug delivery system is administered by intracarotid injection.

19. The method as claimed in claim 16, wherein the tumor cells are selected from the group consisting of head and neck tumor cells, brain tumor cells, skin tumor cells, pancreatic tumor cells, liver tumor cells, and lung tumor cells.

20. The method as claimed in claim 19, wherein the brain tumor cells are glioblastoma multiforme cells.
